(19) United States
(12) Patent Application Publication
(10) Pub. No.: US 2020/0368293 A1
(43) Pub. Date: Nov. 26, 2020

(54) COMPOSITIONS AND METHODS FOR THE TREATMENT OF CANCER

(71) Applicant: Vedanta Biosciences, Inc., Cambridge, MA (US)

(72) Inventor: Bernat Olle, Cambridge, MA (US)

(73) Assignee: Vedanta Biosciences, Inc., Cambridge, MA (US)

(21) Appl. No.: 16/962,274

(22) PCT Filed: Jan. 18, 2019
(86) PCT No.: PCT/US2019/014120

§ 371 Date: Jul. 15, 2020

Related U.S. Application Data
(60) Provisional application No. 62/618,869, filed on Jan. 18, 2018.

(51) Int. Cl. A61K 35/74; A61K 38/19; A61K 38/20; A61K 38/21; C07K 16/28; A61P 35/00

(52) U.S. Cl. CPC A61K 35/74; A61K 38/191; A61K 38/2013; A61K 38/208; A61P 35/00; A61K 38/212; C07K 16/2818; C07K 16/2827; C07K 16/2878; A61K 38/2086

(57) ABSTRACT

Provided herein are compositions, methods, and kits for the treatment of cancer. The disclosure also provides compositions, methods, and kits that suppress adverse events caused by anticancer therapy.

### RELATED APPLICATIONS

This application claims the benefit under 35 U.S.C. § 119(e) of U.S. provisional application No. 62/618,869 filed Jan. 18, 2018. The entire contents of this referenced application are incorporated by reference herein.

### FIELD OF INVENTION

The disclosure relates to compositions and methods for the treatment of cancer. The disclosure also provides methods of treatment that suppress adverse events caused by anticancer therapy.

### BACKGROUND OF THE INVENTION

Anticancer therapy is often associated with adverse events (e.g., toxicities). In the past, adverse events in cancer therapy have not received a lot of attention as the focus of anticancer therapy is to beat the cancer, and save the patients live, at all cost. For instance, traditional chemotherapy comes with many adverse events including nausea and hair loss which have been tolerated as the alternative, succumbing to cancer, is far worse.

Recently, powerful new methods of cancer treatment have been become available in the form of immune checkpoint inhibitors (or checkpoint inhibitors). Checkpoint inhibitors seek to overcome one of cancer's main defenses against a patient's anti-cancer immune response. Cancer therapies that incorporate checkpoint inhibitors have attained improvements in survival rates. However, as checkpoint inhibitors modify the immune response of the patient, the administration of checkpoint inhibitors can result in adverse events. Methods that suppress adverse events associated with cancer checkpoint inhibitor therapy are needed therefore.

### SUMMARY OF THE INVENTION

In one aspect, the disclosure provides methods, compositions, and kits for the treatment of cancer. The disclosure also provides methods, compositions, and kits for the treatment of cancer that suppress adverse events caused by anticancer therapy.

In one aspect, the disclosure provides a method of treatment comprising administering to a subject undergoing anticancer therapy a suppressing agent that suppresses an adverse event caused by the anticancer therapy.

In one aspect, the disclosure provides a method of treatment comprising administering a pharmaceutically effective amount of anticancer therapy to a subject in need thereof and administering to the subject a suppressing agent that suppresses an adverse event caused by the anticancer therapy.

In one aspect, the disclosure provides a method of treatment comprising administering to a subject in need thereof a suppressing agent followed by a pharmaceutically effective amount of anticancer therapy.

In one aspect, the disclosure provides a method of treatment comprising administering to a subject in need thereof a combination of a suppressing agent and a pharmaceutically effective amount of anticancer therapy.

In one aspect, the disclosure provides a method of treatment comprising administering a pharmaceutically effective amount of anticancer therapy to a subject in need thereof, determining if an adverse event occurs in the subject and administering to the subject a suppressing agent that suppresses the adverse event.

In one aspect, the disclosure provides a method of treatment comprising administering a pharmaceutically effective amount of anticancer therapy to a subject in need thereof, determining if an adverse event occurs in the subject, wherein if an adverse event occurs in the subject, administering to the subject a suppressing agent that suppresses the adverse event.

In one aspect, the disclosure provides compositions or combinations of compositions used in any one of the methods disclosed herein.

In one aspect, the disclosure provides kits comprising compositions or combinations of compositions used in any one of the methods disclosed herein.

In some embodiments in any one of the methods, compositions, or kits provided herein, the anticancer therapy comprises the administration of one or more anticancer agents. In some embodiments in any one of the methods, compositions, or kits provided herein, the anticancer agent is a chemotherapy agent. In some embodiments in any one of the methods, compositions, or kits provided herein, the anticancer agent is a cancer immunotherapy agent. In some embodiments in any one of the methods, compositions, or kits provided herein, the cancer immunotherapy agent is an immune checkpoint inhibitor. In some embodiments in any one of the methods, compositions, or kits provided herein, the immune checkpoint inhibitor is a PD-1 inhibitor, PD-L1 inhibitor, or CTLA-4 inhibitor. In some embodiments in any one of the methods, compositions, or kits provided herein, the immune checkpoint inhibitor is a PD-1 inhibitor. In some embodiments in any one of the methods, compositions, or kits provided herein, the PD-1 inhibitor is nivolumab or pembrolizumab. In some embodiments in any one of the methods, compositions, or kits provided herein, the immune checkpoint inhibitor is a PD-L1 inhibitor. In some embodiments in any one of the methods, compositions, or kits provided herein, the PD-L1 inhibitor is atezolizumab, avelumab or durvalumab. In some embodiments in any one of the methods, compositions, or kits provided herein, the immune checkpoint inhibitor is a CTLA-4 inhibitor. In some embodiments in any one of the methods, compositions, or kits provided herein, the CTLA-4 inhibitor is ipilimumab or tremelimumab.

In some embodiments in any one of the methods, compositions, or kits provided herein, the anticancer therapy comprises the administration of one or more cytokines. In some embodiments in any one of the methods, compositions, or kits provided herein, the cytokine is interferon-alpha, tumor necrosis factor, interleukin (IL)-2, IL-12, IL-15, or IL-21.

In some embodiments in any one of the methods, compositions, or kits provided herein, the anticancer therapy comprises the administration of one or more costimulatory agents. In some embodiments in any one of the methods, compositions, or kits provided herein, the costimulatory agent is a CD-28, OX-40, 4-1BB, or CD40 antibody.

In some embodiments in any one of the methods, compositions, or kits provided herein, the anticancer therapy comprises the administration of an anticancer live bacterial product. In some embodiments in any one of the methods, compositions, or kits provided herein, the anticancer live bacterial product induces CD8+ T-cells. In some embodiments in any one of the methods, compositions, or kits provided herein, the anticancer live bacterial product induces Th17 cells. In some embodiments in any one of the methods, compositions, or kits provided herein, the anticancer live bacterial product induces Th1 cells. In some embodiments in any one of the methods, compositions, or kits provided herein, the anticancer live bacterial product comprises bacterial strains of species associated with increased efficacy in anticancer treatment.

In some embodiments in any one of the methods, compositions, or kits provided herein, the anticancer therapy comprises the administration of an immune checkpoint inhibitor and an anticancer live bacterial product. In some embodiments in any one of the methods, compositions, or kits provided herein, the anticancer live bacterial product increases the efficacy of the immune checkpoint inhibitor. In some embodiments in any one of the methods, compositions, or kits provided herein, the anticancer therapy comprises the administration of a PD-1 inhibitor and an anticancer live bacterial product that induces CD8+ T-cells. In some embodiments in any one of the methods, compositions, or kits provided herein, the anticancer therapy comprises the administration of a PD-1 inhibitor and an anticancer live bacterial product that induces Th17 cells. In some embodiments in any one of the methods, compositions, or kits provided herein, the anticancer therapy comprises the administration of a PD-1 inhibitor and an anticancer live bacterial product that induces Th1 cells. In some embodiments in any one of the methods, compositions, or kits provided herein, the anticancer therapy comprises the administration of a PD-1 inhibitor and an anticancer live bacterial product that comprises bacterial strains of species associated with increased efficacy in anticancer treatment with a PD-1 inhibitor.

In some embodiments in any one of the methods, compositions, or kits provided herein, the anticancer therapy comprises the administration of a PD-L1 inhibitor and an anticancer live bacterial product that induces CD8+ T-cells. In some embodiments in any one of the methods, compositions, or kits provided herein, the anticancer therapy comprises the administration of a PD-L1 inhibitor and an anticancer live bacterial product that induces Th17 cells. In some embodiments in any one of the methods, compositions, or kits provided herein, anticancer therapy comprises the administration of a PD-L1 inhibitor and an anticancer live bacterial product that induces Th1 cells. In some embodiments in any one of the methods, compositions, or kits provided herein, the anticancer therapy comprises the administration of a PD-L1 inhibitor and an anticancer live bacterial product that comprises bacterial strains of species associated with increased efficacy in anticancer treatment with a PD-L1 inhibitor.

In some embodiments in any one of the methods, compositions, or kits provided herein, the anticancer therapy comprises the administration of a CTLA-4 inhibitor and an anticancer live bacterial product that induces CD8+ T-cells. In some embodiments in any one of the methods, compositions, or kits provided herein, the anticancer therapy comprises the administration of a CTLA-4 inhibitor and an anticancer live bacterial product that induces Th17 cells. In some embodiments in any one of the methods, compositions, or kits provided herein, the anticancer therapy comprises the administration of a CTLA-4 inhibitor and an anticancer live bacterial product that induces Th1 cells. In some embodiments in any one of the methods, compositions, or kits provided herein, the anticancer therapy comprises the administration of a CTLA-4 inhibitor and an anticancer live bacterial product that comprises bacterial strains of species associated with increased efficacy in anticancer treatment with a CTLA-4 inhibitor.

In some embodiments in any one of the methods, compositions, or kits provided herein, the methods include the administration of a suppressing agent. In some embodiments in any one of the methods, compositions, or kits provided herein, the suppressing agent is an agent that suppresses the immune response. In some embodiments in any one of the methods, compositions, or kits provided herein, the suppressing agent is a suppressing live bacterial product. In some embodiments in any one of the methods, compositions, or kits provided herein, the suppressing live bacterial product induces regulatory T cells. In some embodiments in any one of the methods, compositions, or kits provided herein, the suppressing live bacterial product comprises bacterial strains belonging to Clostridium cluster XIVa and/or Clostridium cluster IV. In some embodiments in any one of the methods, compositions, or kits provided herein, the suppressing live bacterial product comprises bacterial strains belonging to Clostridium cluster XIVa. In some embodiments in any one of the methods, compositions, or kits provided herein, the suppressing live bacterial product is VE-202.

In some embodiments in any one of the methods provided herein, the methods include the suppression of an adverse event. In some embodiments in any one of the methods provided herein, the adverse event is an undesired immune response. In some embodiments in any one of the methods provided herein, the adverse event is colitis. In some embodiments in any one of the methods provided herein, the adverse event is dermatological toxicity. In some embodiments in any one of the methods provided herein, the adverse event is diarrhea. In some embodiments in any one of the methods provided herein, the adverse event is hepatotoxicity. In some embodiments in any one of the methods provided herein, the adverse event is hypophysitis. In some embodiments in any one of the methods provided herein, the adverse event is autoimmune thyroid disease.

In some embodiments in any one of the methods provided herein, the suppressing agent is administered after the occurrence of the adverse event. In some embodiments in any one of the methods provided herein, the suppressing agent is administered prior to the occurrence of the adverse event. In some embodiments in any one of the methods provided herein, the method further comprises repeating the anticancer therapy. In some embodiments in any one of the methods provided herein, the method further comprises repeating the administration of a suppression agent. In some embodiments in any one of the methods provided herein, the method further comprises repeating the anticancer therapy and repeating the administration of a suppression agent. In some embodiments in any one of the methods provided herein, the method further comprises determining if an adverse event occurs. In some embodiments in any one of the methods provided herein, the subject is treated with antibiotics prior to administration of the suppressing agent. In some embodiments in any one of the methods provided herein, multiple doses of the suppressing agent are administered. In some embodiments in any one of the methods provided herein, the suppressing agent is administered after the completion of one round of anticancer therapy. In some embodiments in any one of the methods provided herein, the suppressing agent is administered at least one week, at least two weeks, at least three weeks, or at least twelve weeks after the completion of one round of anticancer therapy. In some embodiments in any one of the methods provided herein, the suppressing agent is administered prior to the completion of one round of anticancer therapy. In some embodiments in any one of the methods provided herein, the suppressing agent is administered at least one week, at least two weeks, at least three weeks, or at least twelve weeks after the initiation of the anticancer therapy.

In some embodiments in any one of the methods provided herein, determining if an adverse event occurs includes determining if IL-17 is increased in serum of the subject. In some embodiments in any one of the methods provided herein, determining if an adverse event occurs includes testing for eosinophilia.

In some embodiments in any one of the methods provided herein, the subject is being treated for melanoma, non-small cell lung cancer (NSCLC), Hodgkin's lymphoma, head and neck cancer, renal cell cancer, bladder cancer, or Merkel cell carcinoma.

In some embodiments in any one of the methods provided herein, the method further comprises administering a steroid as a suppressing agent. In some embodiments in any one of the methods provided herein, the steroid is prednisone. In some embodiments in any one of the methods provided herein, the suppressing agent comprises infliximab. In some embodiments in any one of the methods provided herein, the method further comprises administering infliximab as a second suppressing agent.

In one aspect, the disclosure provides a composition comprising any one of the anticancer agents provided herein and any one of the suppressing agents provided herein.

In one aspect, the disclosure provides a kit comprising an agent for detecting a biomarker for an adverse event and any one of suppressing agents provided herein.

In one aspect, the disclosure provides a kit comprising any one of the anticancer agents provided herein, an agent for detecting a biomarker for an adverse event and any one of suppressing agents provided herein.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The accompanying drawings are not intended to be drawn to scale. The figures are illustrative only and are not required for enablement of the disclosure. For purposes of clarity, not every component may be labeled in every drawing. In the drawings:

FIGS. 1A-1K show schematic diagrams of various example embodiments of anticancer therapeutic regimens including anticancer therapy and a suppressing agent.

### DETAILED DESCRIPTION OF THE INVENTION

Administration of anticancer therapies frequently results in adverse effects (e.g., side effects, toxicities) of varying severity. Such adverse effects can lead to the discontinuation of the anticancer therapy. Described herein are methods, compositions, and kits for the treatment of cancer. Also provided are methods, compositions, and kits for the treatment of adverse events caused by anticancer therapy, as well as methods, compositions, and kits for the treatment of cancer including the suppression of adverse events caused by the anticancer therapy.

Aspects of the present disclosure relate to methods involving administering to a subject undergoing anticancer therapy a suppressing agent that suppresses an adverse event caused by the anticancer therapy. Aspects relate to methods involving administering a pharmaceutically effective amount of anticancer therapy to a subject in need thereof and administering a suppressing agent that suppresses an adverse event, for example if an adverse event occurs. Aspects relate to methods involving administering a pharmaceutically effective amount of a suppressing agent prior to or concurrently with an anticancer therapy. Also provided herein are compositions, combinations of anticancer therapies, and kits for use in any of the methods disclosed herein.

### Anticancer Therapy

Aspects of the present disclosure provide methods, compositions, and kits for use in anticancer therapy. As used herein, the term “anticancer therapy” refers to any therapeutic regimen that aims to reduce or eliminate cancer, slow the progression of cancer, prevent or reduce the risk of cancer metastasis, and/or reduce or prevent any one or more symptoms associated with cancer. The anticancer therapies described herein involve the administering anticancer therapies to a subject, e.g., a subject having cancer or at risk of having cancer.

As will be appreciated by one of skill in the art, anticancer therapies may involve administration to the subject of one or more anticancer agents, surgery, radiation therapy, or a combination thereof.

In some embodiments, the anticancer therapy involves administration of one or more (e.g., 2, 3, 4, 5, or more) anticancer agents. In some embodiments, the anticancer therapy may involve a single administration of a single anticancer agent. In some embodiments, the anticancer therapy may involve a single administration of multiple anticancer agents. In some embodiments, the anticancer therapy may involve multiple administrations of a single anticancer agent, or multiple administrations of different anticancer agents. Anticancer agents include any therapeutic (e.g., small molecule, antibody, live bacterial product) that aims to target and eliminate cancer cells in the subject.

In some embodiments, the anticancer agent is a chemotherapy agent. As used herein, a chemotherapy agent refers to a molecule (e.g., drug) that specifically or preferentially kills cancer cells or prevents the proliferation of cancer cells. Chemotherapy agents can generally be categorized based on the molecular target of the chemotherapy agent, the mechanism of action, and/or the structure of the agent. In some embodiments, the chemotherapy agent is an alkylating agent, a plant alkaloid, an antitumor antibiotic, an antimetabolite, a topoisomerase inhibitor, or other antineoplastic agent.

Examples of chemotherapy agents include, without limitation, Methotrexate, Paclitaxel, Brentuximab Vedotin, Doxorubicin, 5-FU (fluorouracil), Everolimus, Pemetrexed, Melphalan, Pamidronate, Anastrozole, Exemestane, Nelarabine, Ofatumumab, Bevacizumab, Belinostat, Tositumomab, Carmustine, Bleomycin, Blinatumomab, Bosutinib, Busulfan, Alemtuzumab, Irinotecan, Vandetanib, Bicalutamide, Lomustine, Daunorubicin, Clofarabine, Cabozantinib, Dactinomycin, Cobimetinib, Ramucirumab, Cytarabine, Cytoxan, Cyclophosphamide, Decitabine, Dexamethasone, Docetaxel, Hydroxyurea, Decarbazine, Leuprolide, epirubicin, oxaliplatin, Asparaginase, Estramustine, Cetuximab, Vismodegib, Asparaginase Erwinia chrysanthemi, Amifostine, Etoposide, Flutamide, Toremifene, Panobinostat, Fulvestrant, Letrozole, Degarelix, Fludarabine, Pralatrexate, floxuridine, Obinutuzumab, Gemcitabine, Afatinib, Imatinib Mesylate, Carmustine wafer, Eribulin, Trastuzumab, Altretamine, Topotecan, Palbociclib, Ponatinib, Idarubicin, Ifosfamide, Ibrutinib, Axitinib, Interferon alfa-2a, Gefitinib, Romidepsin, Ixabepilone, Ruxolitinib, Cabazitaxel, Ado-trastuzumab Emtansine, Pembrolizumab, Carfilzomib, Lenvatinib, Chlorambucil, Sargramostim, Cladribine, Trifluridine and Tipiracil, Olaparib, Mitotane, Vincristine, Procarbazine, Megestrol, Trametinib, Mesna, Strontium-89 Chloride, Mechlorethamine, Mitomycin, Gemtuzumab Ozogamicin, Vinorelbine, Cyclophosphamide, filgrastim, pegfilgrastim, Sorafenib, nilutamide, Pentostatin, Mitoxantrone, Sonidegib, Pegaspargase, Denileukin Diftitox, Nivolumab, Alitretinoin, Carboplatin, Pertuzumab, Cisplatin, Pomalidomide, Prednisone, Aldesleukin, Mercaptopurine, Zoledronic acid, Lenalidomide, Rituximab, Octreotide, Tamoxifen, Dasatinib, Regorafenib, Sunitinib, Peginterferon Alfa-2b, Siltuximab, Omacetaxine, Thioguanine, Dabrafenib, Erlotinib, Bexarotene, Decarbazine, Docetaxel, Temozolomide, Thiotepa, Thalidomide, BCG, Temsirolimus, Bendamustine hydrochloride, Triptorelin, Arsenic trioxide, lapatinib, Dinutuximab, Valrubicin Intravesical, Histrelin, Panitumumab, Vinblastine, Bortezomib, Tretinoin, Azacitidine, Pazopanib, Teniposide, Leucovorin, Crizotinib, Capecitabine, Enzalutamide, Ipilimumab, Trabectedin, Ziv-aflibercept, Streptozocin, Vemurafenib, Ibritumomab Tiuxetan, Goserelin, Vorinostat, Idelalisib, Ceritinib, and Abiraterone.

### Cancer Immunotherapy Agents

In some embodiments, the anticancer therapy involves administration of one or more (e.g., 2, 3, 4, 5, or more) anticancer agents. In some embodiments, the anticancer agent is a cancer immunotherapy agent. Cancer immunotherapy agents aim to harness an immune response or produce an immune response to a cancer or cancer cell to specifically attack the cancer or cancer cells, thereby eliminating or reducing the cancer. Examples of cancer immunotherapy agents include immune checkpoint inhibitors, monoclonal antibodies, cytokines, cancer vaccines, and cytotoxic T cells expressing chimeric antigen receptors (CAR-T cells).

In some embodiments, the cancer immunotherapy agent is an immune checkpoint inhibitor (also referred to as “checkpoint inhibitor”). Immune checkpoints are regulatory pathways within the immune system that are involved in maintaining immune homeostasis (e.g., self-tolerance, modulating the duration and extent of an immune response) to minimize cellular damage due to aberrant immune responses. Inhibitors of immune checkpoints, herein referred to as “immune checkpoint inhibitors,” specifically inhibit immune checkpoints and may have a stimulatory or inhibitory effect on the immune response. Without wishing to be bound by any particular theory, it is thought in art that different cancers and tumors may manipulate immune checkpoints to evade detection and/or modulate the immune response.

In some embodiments, the immune checkpoint inhibitor is a PD-1 inhibitor, PD-L1 inhibitor, or CTLA-4 inhibitor. (See e.g., Vesely M D, Annu Rev Immunol (2011) 29:235-271; Pardoll, Nature Reviews Cancer (2012) 12, 252-264). In some embodiments of the compositions provided herein, the immune checkpoint inhibitor is a PD-1 inhibitor, PD-L-1 inhibitor, CTLA-4 inhibitor, IDO1 inhibitor, LAG3 inhibitor, or TIM3 inhibitor.

In some embodiments, the immune checkpoint inhibitor is a PD-1 inhibitor. In some embodiments, the PD-1 inhibitor is nivolumab. In some embodiments, the PD-1 inhibitor is pembrolizumab.

In some embodiments, the immune checkpoint inhibitor is a PD-L1 inhibitor. In some embodiments, the PD-L1 inhibitor is atezolizumab. In some embodiments, the PD-L1 inhibitor is avelumab. In some embodiments, the PD-L1 inhibitor is durvalumab.

In some embodiments, the immune checkpoint inhibitor is a CTLA-4 inhibitor. In some embodiments, the CTLA-4 inhibitor is an anti-CTLA-4 antibody. Examples of anti-CTLA-4 antibodies include, without limitation, ipilimumab, tremelimumab (CP-675,206), 9H10, 4F10, and 9D9. In some embodiments, the CTLA-4 inhibitor is ipilimumab. In some embodiments, the CTLA-4 inhibitor is tremelimumab.

It should further be appreciated that multiple immune checkpoint inhibitors may be used in the methods, compositions, and kits disclosed herein. For instance, in a non-limiting example, the methods described herein include the administration of both a PD-1 inhibitor and a CTLA-4 inhibitor.

In some embodiments, the cancer immunotherapy agent is a cytokine. Cytokines are signaling molecules naturally produced by cells that have immunomodulatory effects. Administration of exogenous cytokines has the capability of modulating the immune response to achieve a desired effect, such as the specific targeting of cancer cells. Cytokines may be administered alone as a cancer immunotherapy or in combination with one or more cancer therapies to enhance the therapeutic effect of the cancer therapy. In some embodiments, the cancer immunotherapy agent is a cytokine that is able to modulate the immune response to reduce or eliminate cancer cells. Examples of cytokines for use as cancer immunotherapy agents include type I interferons (e.g., IFNα), IFNγ, tumor necrosis factor (TNF, TNFα, TNF-alpha), and interleukins (e.g., IL-2, IL-12, IL-15, and IL-21).

In some embodiments, the cancer immunotherapy agent comprises an anticancer vaccine (also referred to herein as a cancer vaccine). Cancer vaccines generally act to increase an immune response to cancer cells. For example, cancer vaccines include cancer antigen(s) that act to induce or stimulate an immune response against cells bearing the cancer antigen(s). The immune response induced or stimulated can include an antibody (humoral) immune response and/or a T-cell (cell-mediated) immune response. CD8+ T-cells can differentiate into cytotoxic T-cells that kill target cells bearing the antigen recognized by CD8+ T-cells. Induction of CD8+ T-cells can, therefore, enhance the immune response to cancer antigens provided in a cancer vaccine.

In some embodiments, the cancer vaccine is a dendritic cell vaccine. Dendritic cell vaccines may involve harvesting cells from a subject, specifically producing or proliferating dendritic cells from the harvested cells ex vivo, loading the dendritic cells with cancer antigens and/or activating the dendritic cells, and administering the dendritic cells to the subject. In some embodiments, the dendritic cells of the dendritic cell vaccine are autologous cells, meaning the dendritic cells were harvested and re-administered to the same subject. In some embodiments, the dendritic cells of the dendritic cell vaccine are allogeneic cells, meaning the dendritic cells were harvested from one subject (e.g., the donor) and administered to a different subject (e.g., the recipient).

In some embodiments, the cancer immunotherapy agent comprises adoptive cell transfer therapy. In general, adoptive cell transfer therapy involves harvesting cells from a subject, specifically producing or expanding a specific cell population, optionally activating the cells, and administering the expanded cells to the subject. In some embodiments, the desired cells are immune cells capable of killing or eliminating cancer cells.

In some embodiments, the adoptive cell transfer therapy uses engineered T-cell receptors or chimeric antigen receptors, which may be referred to as CAR-T therapy. CAR-T cells include T-cells taken from a subject that are genetically engineered to express chimeric antigen receptors (CARs) on the cell surface. The CAR-T cell receptors are designed to recognize a specific antigen on cancer cells (e.g., a cancer antigen). After the CAR-T cells are infused into the subject, the CAR-T cells recognize and kill cancer cells that express the specific antigen on their surfaces. In some embodiments, the CAR-T cells are autologous cells, meaning the T cells were harvested and re-administered to the same subject. In some embodiments, the CAR-T cells are CD8+ T cells. In some embodiments, the CAR-T cells are allogeneic cells, meaning the T cells were harvested from one subject (e.g., the donor) and administered to a different subject (e.g., the recipient).

Examples of cancer antigens that may be targeted by CAR-T cells are known in the art, and selection of a cancer antigen for targeting will depend on factors such as the cancer that is being targeted.

In some embodiments, the anticancer therapy involves administering one or more costimulatory agents. In some embodiments, the costimulatory agent is a molecule that targets one or more costimulatory molecules, thereby modulating the immune response. In some embodiments, the costimulatory agent enhances an anticancer immune response, for example, by preventing the downregulation of an immune response. A costimulatory agent may be administered alone in a cancer therapy or in combination with one or more cancer therapies to enhance the therapeutic effect of the cancer therapy. In some embodiments, the costimulatory agent is an antibody that targets CD-28, OX-40, 4-1BB, or CD40.

### Anticancer Live Bacterial Products

Aspects of the present disclosure provide methods, compositions, and kits for use in anticancer therapy. In some embodiments, the anticancer therapy comprises the administration of an anticancer live biotherapeutic product (e.g., live bacterial product, live viral product, live fungal product). In some embodiments, the anticancer therapy comprises the administration of an anticancer live bacterial product. As described herein, a live bacterial product (also referred to as bacterial cocktail, live bacterial consortium, or bacterial consortium) comprises one or more bacterial strains. The bacterial composition of the anticancer live bacterial product is selected based on the ability of the live bacterial product to induce or stimulate a desired response when administered to a subject (e.g., a cancer patient). In some embodiments, the anticancer live bacterial product induces or stimulates an anticancer effect (e.g., inhibition or cytotoxicity of cancer cells) when administered to the subject. In some embodiments, the anticancer live bacterial product induces or stimulates an immune response that provides an anticancer effect when administered to the subject.

As will be appreciated by one of skill in the art, one or more bacterial strains may be selected and combined in a live bacterial product. As described herein, an anticancer live bacterial product may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more bacterial strains. The ability of the specific combination of bacterial strains of the live bacterial product to induce an anticancer effect can be assessed using any of method known in the art, e.g., in vitro assays for example using cell culture, or in vivo studies. In some embodiments, the anticancer live bacterial product induces a specific immune cell population (e.g., CD8+ T-cells, Th17, Th1 cells). The abundance of a specific population of cells (e.g., CD8+ T-cells, Th17, Th1 cells) can be assessed by any method known in the art, for example by detecting a cellular marker indicative of the cell type, assessing a direct or indirect activity of the cell type, and/or by measuring the production of one or more cytokines produced by the specific cell type.

In some embodiments, the anticancer live bacterial product induces CD8+ T-cells (or “CD8+ T cells”). As will be appreciated by one of ordinary skill in the art, a combination of bacterial strains may be selected and combined to produce an anticancer live bacterial product that induces CD8+ T-cells. Non-limiting examples of live bacterial products that induce CD8+ T-cells, can be found in PCT Publication WO2018/117263 which is herein incorporated by reference in its entirety. In some embodiments, the anticancer live bacterial product that induces CD8+ T-cells comprises one or more bacteria selected from:

1) Phascolarctobacterium faecium or Phascolarctobacterium sp. CAG:207,

2) Fusobacterium ulcerans or Fusobacterium varium,

3) Bacteroides dorei or Bacteroides fluxus,

4) Bacteroides uniformis or Bacteroides sp. D20,

5) Subdoligranulum sp., Ruthenibacterium lactatiformans, Ruminococcaceae bacterium cv2 or Gemminger formicilis,

6) Paraprevotella xylaniphila,

7) Parabacteroides johnsonii,

8) Alistipes sp., Alistipes timonensis, or Alistipes senegalensis,

9) Parabacteroides gordonii or Parabacteroides sp. HGS0025,

10) Eubacterum limosum, and

11) Parabacteroides distasonis or Parabacteroides sp. CAG:2.

In some embodiments, the anticancer live bacterial product contains one or more bacterial strains of each of the eleven recited groups.

Assessment of the extent of induction of proliferation or accumulation of CD8+ T-cells can be performed using any method known in the art, for example by determining the number of CD8+ T-cells prior to and after administration of the live bacterial product, or by measurement of CD8+ activity, such as cytotoxicity, release of cytotoxins, production of one or more cytokines indicative of CD8+ T cells.

In some embodiments, the anticancer live bacterial product induces Th17 cells. In some embodiments, the anticancer live bacterial product induces the proliferation and/or accumulation of Th17 cells. As will be appreciated, a combination of bacterial strains may be selected and combined to produce an anticancer live bacterial product that induces Th17 cells. Non-limiting examples of live bacterial products, including single bacterial species, that induce Th17 cells, can be found in PCT Publication WO2015/156419 as well as Atarashi et al. Cell (2015)163(2): 367-380; Tan et al. PNAS (2016), 113: 8141; Wu et al. Nature Medicine (2009)15: 1016; PCT publication WO2017/089794; PCT publication WO2017/089795; and PCT publication WO2017/085518, which are all herein incorporated by reference in their entirety. In some embodiments, the anticancer live bacterial product that induces Th17 cells comprises one or more bacteria selected from Clostridium symbiosum, Clostridium hathewayi, Clostridium citroniae, Clostridium bolteae, Ruminococcus sp. M-1, Ruminococcus gnavus, Blautia sp. canine oral taxon 143, Anaerostipes caccae, Clostridium lactatifermentans, Coprobacillus cateniformis, Clostridium ramosum, cf., Clostridium sp. MLG055, Clostridium innocuum, Eubacterium desmolans, Clostridium orbiscindens, Ruminococcus sp. 16442, Anaerotruncus colihominis, Bacteroides dorei, Bifidobacterium pseudolongum subsp. Pseudolongum, and Bifidobacterium breve (See also PCT Publication WO2015/156419 as well as Atarashi et al. Cell (2015)163(2): 367-380). In some embodiments, the anticancer live bacterial product that induces Th17 cells comprises Erysipelatoclostridium ramosum (See, e.g., also PCT publication WO2017/089794), Eubacterium contortum (See, e.g., also PCT publication WO2017/089795), Enterococcus faecium (See, e.g., also PCT publication WO2017/085518).

Assessment of the extent of induction of proliferation or accumulation of Th17 cells can be performed using any method known in the art, for example by determining the number of Th17 cells prior and after administration of the live bacterial product, or by measurement of Th17 activity, such as expression of at least one of ROR-gamma-t, IL-17 A, IL-7F, IL-22, IL-23, IL-23R, CD 161, and CCR6.

In some embodiments, the anticancer live bacterial product induces Th1 cells. As will be appreciated by one of ordinary skill in the art, a combination of bacterial strains may be selected and combined to produce an anticancer live bacterial product that induces Th1 cells. It has been demonstrated that specific bacterial species may be associated with the induction of Th1 cells. Non-limiting examples of live bacterial products, including single bacterial strains, that induce Th1 cells include Bacteroides spp. (e.g., B. fragilis, B. thetaiotaomicron) and Burkholderiales (See, e.g., Pitt et al. Oncoimmunology (2017) 6(1)), Klebsiella species, and related bacterial species that have also been shown to induce Th1 cells (See e.g., Atarashi et al. Science (2017) 358 (6361) and PCT publication WO2018/084172, which are all herein incorporated by reference in their entirety).

Assessment of the extent of induction of proliferation or accumulation of Th1 cells can be performed using any method known in the art, for example by determining the number of Th1 cells prior to and after administration of the live bacterial product, or by measurement of Th1 activity, such as expression of IFNγ, TN93, and/or IL-2.

It has been found that the population of an individual's gut microbiome may influence the individual's response to cancer treatment. In particular, the presence of certain bacterial strains has been associated with improved efficacy in anti-cancer therapy. In some embodiments, the anticancer live bacterial product of the methods, compositions, and kits provided herein, includes bacterial strains that are associated with improved efficacy in anticancer therapy (also referred to as “increased efficacy” or “enhanced efficacy”). In some embodiments, one or more bacterial strains of the anticancer live bacterial product may be selected for exhibiting a desired property, such as enhancing the effects of another anticancer therapy. Bacterial strains that are associated with improved efficacy in anticancer therapy include Bacteroides species (e.g., B. fragilis, B. thetaiotaomicron) and Burkholderiales species, which are associated with enhanced efficacy of an anticancer therapy involving administration of a CTLA-4 inhibitor; See, e.g., Vétizou et al. Science (2015) 350(6264): 1079-1084; Pitt et al. Oncoimmunology (2017) 6(1)) and PCT publications WO2015/075688 and WO2016/063263. Certain Bifidobacterium species are associated with enhanced efficacy of an anticancer therapy involving administration of a PD-L1 inhibitor; See, e.g., Sivan et al. Science (2015) 350(6264)) and PCT publication WO2016/196605. Furthermore, Ruminococcaceae species, within the Clostridiales order, are associated with enhanced efficacy of an anticancer therapy involving administration of a PD-L1 inhibitor; See Wargo, J. “Diversity and composition of the gut microbiome influence response to anti-PD-1 immune checkpoint therapy in patients with metastatic melanoma” ASCO 2017 poster DOI: 10.1200/JCO.2017.35.15_supp1.3008 Journal of Clinical Oncology 35, no. 15_suppl (May 2017) 3008-3008)). In addition, enhanced efficacy of treatment with the CTLA-4 inhibitor ipilimumab has also been associated with the presence of Faecalibacterium and other Firmicutes; see, Chaput et al. Ann. Oncol. (2017) 28(6): 1368-1379 and PCT publication WO2018/172483. Additionally, it was found that subjects that responded positively to treatment with the PD-1 inhibitor nivolumab in combination with the CTLA-4 inhibitor ipilimumab had microbiomes enriched with Bacteroides thetaiotaomicron, Alistipes shahii, Holdemania, Faecalibacterium and Firmicutes, such as Ruminococcaceae, and Streptococcus mutans; whereas individuals that responded to treatment with the PD-1 inhibitor pembrolizumab had microbiomes enriched with Dorea formicigenerans; See, e.g., Frankel et al. Neoplasia, 2017 volume 19, pages 848-855, and Frankel. et al. Metagenomic shotgun sequencing to identify specific human gut microbes associated with immune checkpoint therapy efficacy in melanoma patients. (Abstract 9516 and Poster Board 124; ASCO 2017) A. E. Frankel, T. W. Froehlich, J. Kim, L. A. Coughlin, Y. Xie, E. P. Frenkel, A. Y), see also PCT publication WO2018/222969. Akkermansia species, Enterococcus hirae, and Alistipes spp. were associated with an increased efficacy with PD-1 inhibition; See, Routy et al. Science (2018) 359(6371): 91-97 and PCT publication WO2018/115519. Faecalibacterium spp and Ruminococcaceae were also associated with enhanced treatment with a PD-1 inhibitor; See Gopalakrishnan et al. Science (2017) 259(6371): 97-103 and PCT publication WO2018/064165. Furthermore, Bifidobacterium longum, Collinsella aerofaciens, and Enterococcus faecium are associated with enhanced efficacy in anti-PD1 and anti-PD-L1 efficacy; See Matson et al. Science (2018), 359; 104-108. Additional bacterial strains associated with enhanced efficacy in anticancer therapy, including anticancer therapy with checkpoint inhibitors, are Bacillus Calmette-Guerin (See PCT application WO2018/112360), Parabacteroides goldsteinii (See PCT application WO2018/112363), and Enterococcus gallinarum (See PTC publications WO2018/215782 and WO2017/085520). Thus, in some embodiments, the anticancer live bacterial product includes bacterial strains that are associated with improved efficacy in anticancer therapy. In some embodiments, the anticancer live bacterial product includes one or more bacterial strains of species selected from the group consisting of Bacteroides sp., Burkholderia sp., Bifidobacterium sp., Ruminococcaceae sp., Faecalibacterium sp., Bacteroides thetaiotaomicron, Alistipes shahii, Holdemania sp., Streptococcus mutans, Dorea formicigenerans, Akkermansia sp., Enterococcus hirae, Alistipes sp. Bifidobacterium longum, Collinsella aerofaciens, Enterococcus faecium, Bacillus Calmette-Guerin, Parabacteroides goldsteinii, and Enterococcus gallinarum. It should be appreciated that each of the references cited in this paragraph are all herein incorporated by reference in their entirety.

In some embodiments, the disclosure includes combinations of anticancer live bacterial products, including anticancer live bacterial products associated with inhibitor anticancer therapy. Thus, for instance, live bacterial products that induce Th17 may be combined with live bacterial products that induce CD8+ T-cells, and/or bacterial strains that are associated with improved efficacy in anticancer therapy (e.g., Bacteroides sp., Burkholderia sp., Bifidobacterium sp., Ruminococcaceae sp., Faecalibacterium sp., Bacteroides thetaiotaomicron, Alistipes shahii, Holdemania sp., Streptococcus mutans, Dorea formicigenerans, Akkermansia sp., Enterococcus hirae, Alistipes sp. Bifidobacterium longum, Collinsella aerofaciens, Enterococcus faecium, Bacillus Calmette-Guerin, Parabacteroides goldsteinii, and Enterococcus gallinarum).

In some embodiments, the subject may be administered one or more doses of an antibiotic prior to or concurrently with anticancer live bacterial products. Antibiotics may be administered for a variety of reasons. For instance, antibiotics may be administered to remove bacterial species from the colon and/or intestine prior to administration of the anticancer live bacterial products provided herein. In some embodiments, antibiotics are administered to increase the ability of the bacterial strains of the anticancer live bacterial products to engraft in the colon and/or intestine. Antibiotics may also be administered to suppress unwanted infections in the case of anticancer therapy. In some instances, antibiotics may be administered as a treatment method for an infectious disease.

In some embodiments, the subject is administered a single dose of an antibiotic prior to the anticancer live bacterial product. In some embodiments, the subject is administered multiple doses of an antibiotic prior to the anticancer live bacterial product. In some embodiments, the subject is administered at least 2, 3, 4, 5 or more doses of an antibiotic prior to the anticancer live bacterial product. In some embodiments, the subject is administered a dose of an antibiotic at substantially the same time as the anticancer live bacterial product. Examples of antibiotics that can be administered include, without limitation, kanamycin, gentamicin, colistin, metronidazole, vancomycin, clindamycin, fidaxomicin, and cefoperazone.

### Combination Anticancer Therapies

Also within the scope of the present disclosure are combination anticancer therapies. In some embodiments, the anticancer therapy involves multiple (e.g., at least 2, 3, 4, 5 or more) anticancer agents. In some embodiments, the anticancer therapy involves multiple (e.g., at least 2, 3, 4, 5 or more) cancer immunotherapy agents. In some embodiments, the anticancer therapy involves at least one anticancer agent and at least one cancer immunotherapy agent. In some embodiments, the anticancer therapy involves at least one cancer immunotherapy agent and at least one anticancer live bacterial product.

As will be appreciated by one of skill in the art, the anticancer therapies of the combination of anticancer therapies may be administered to the subject concomitantly. In some embodiments, the combination of anticancer therapies is administered to the subject in more than one composition. In some embodiments, the anticancer therapies of the combination of anticancer therapies are administered simultaneously to the subject. In some embodiments, the combination of anticancer therapies is administered to the subject in the same composition. In some embodiments, the anticancer therapies of the combination of anticancer therapies are administered sequentially to the subject.

Aspects of the present disclosure provide methods, compositions, and kits, that include combination anticancer therapies comprising one or more immune checkpoint inhibitors and one or more anticancer live bacterial products. In some embodiments, the disclosure provides combination anticancer therapies comprising the administration of an immune checkpoint inhibitor and an anticancer live bacterial product. In some embodiments, the anticancer therapy comprises the administration of a PD-1 inhibitor and an anticancer live bacterial product that induces CD8+ T-cells. In some embodiments, the anticancer therapy comprises the administration of a PD-1 inhibitor and an anticancer live bacterial product that induces Th17 cells. In some embodiments, the anticancer therapy comprises the administration of a PD-1 inhibitor and an anticancer live bacterial product that induces Th1 cells. In some embodiments, the anticancer therapy comprises the administration of a PD-1 inhibitor and an anticancer live bacterial product, wherein the anticancer live bacterial product includes bacterial strains of species associated with increased efficacy in anticancer treatment. In some embodiments, the PD-1 inhibitor is nivolumab or pembrolizumab.

In some embodiments, the anticancer therapy comprises the administration of a PD-L1 inhibitor and an anticancer live bacterial product that induces CD8+ T-cells. In some embodiments, the anticancer therapy comprises the administration of a PD-L1 inhibitor and an anticancer live bacterial product that induces Th17 cells. In some embodiments, the anticancer therapy comprises the administration of a PD-L1 inhibitor and an anticancer live bacterial product that induces Th1 cells. In some embodiments, the anticancer therapy comprises the administration of a PD-L1 inhibitor and an anticancer live bacterial product, wherein the anticancer live bacterial product includes bacterial strains of species associated with increased efficacy in anticancer treatment. In some embodiments, the PD-L1 inhibitor is atezolizumab, avelumab, or durvalumab.

In some embodiments, the anticancer therapy comprises the administration of a CTLA-4 inhibitor and an anticancer live bacterial product that induces CD8+ T-cells. In some embodiments, the anticancer therapy comprises the administration of a CTLA-4 inhibitor and an anticancer live bacterial product that induces Th17 cells. In some embodiments, the anticancer therapy comprises the administration of a CTLA-4 inhibitor and an anticancer live bacterial product that induces Th1 cells. In some embodiments, the anticancer therapy comprises the administration of a CTLA-4 inhibitor and an anticancer live bacterial product wherein the anticancer live bacterial product includes bacterial strains of species associated with increased efficacy in anticancer treatment. In some embodiments, the CTLA-4 inhibitor is ipilimumab or tremelimumab.

In some embodiments, the anticancer therapy comprises the administration of more than one checkpoint inhibitor (e.g., a CTLA-4 inhibitor and a PD-1 inhibitor) and an anticancer live bacterial product that induces CD8+ T-cells. In some embodiments, the anticancer therapy comprises the administration of more than one checkpoint inhibitor (e.g., a CTLA-4 inhibitor and a PD-1 inhibitor) and an anticancer live bacterial product that induces Th17 cells. In some embodiments, the anticancer therapy comprises the administration of more than one checkpoint inhibitor (e.g., a CTLA-4 inhibitor and a PD-1 inhibitor) and an anticancer live bacterial product that induces Th1 cells. In some embodiments, the anticancer therapy comprises the administration of more than one checkpoint inhibitor (e.g., a CTLA-4 inhibitor and a PD-1 inhibitor) and an anticancer live bacterial product, wherein the anticancer live bacterial product includes bacterial strains of species associated with increased efficacy in anticancer treatment.

In some embodiments, one component of the combination anticancer therapy enhances the efficacy of the other component(s) of the combination. In some embodiments, the anticancer live bacterial product, or one or more bacterial strains or species therein, enhances the efficacy of the other component(s) of the combination, for example an immune checkpoint inhibitor. Bacterial strains that are associated with improved efficacy in anticancer therapy with an immune checkpoint inhibitor include bacterial strains of species selected from the group consisting of Bacteroides sp., Burkholderia sp., Bifidobacterium sp., Ruminococcaceae sp., Faecalibacterium sp., Bacteroides thetaiotaomicron, Alistipes shahii, Holdemania sp., Streptococcus mutans, Dorea formicigenerans, Akkermansia sp., Enterococcus hirae, Alistipes sp., Bifidobacterium longum, Collinsella aerofaciens, Enterococcus faecium, Bacillus Calmette-Guerin, Parabacteroides goldsteinii, and Enterococcus gallinarum. Thus, in some embodiments of the methods, compositions, and kits provided herein, the anticancer therapy includes the administration of an immune checkpoint inhibitor and one or more bacterial strains of species selected from the group consisting of Bacteroides sp., Burkholderia sp., Bifidobacterium sp., Ruminococcaceae sp., Faecalibacterium sp., Bacteroides thetaiotaomicron, Alistipes shahii, Holdemania sp., Streptococcus mutans, Dorea formicigenerans, Akkermansia sp., Enterococcus hirae, Alistipes sp., Bifidobacterium longum, Collinsella aerofaciens, Enterococcus faecium, Bacillus Calmette-Guerin, Parabacteroides goldsteinii, and Enterococcus gallinarum. In some embodiments of the methods, compositions, and kits provided herein, the anticancer therapy includes the administration of a PD-1 inhibitor and one or more bacterial strains of species selected from the group consisting of Bacteroides sp., Burkholderia sp., Bifidobacterium sp., Ruminococcaceae sp., Faecalibacterium sp., Bacteroides thetaiotaomicron, Alistipes shahii, Holdemania sp., Streptococcus mutans, Dorea formicigenerans, Akkermansia sp., Enterococcus hirae, Alistipes sp., Bifidobacterium longum, Collinsella aerofaciens, Enterococcus faecium, Bacillus Calmette-Guerin, Parabacteroides goldsteinii, and Enterococcus gallinarum. In some embodiments, the PD-1 inhibitor is nivolumab or pembrolizumab. In some embodiments of the methods, compositions, and kits provided herein, the anticancer therapy includes the administration of a PD-L1 inhibitor and one or more bacterial strains of species selected from the group consisting of Bacteroides sp., Burkholderia sp., Bifidobacterium sp., Ruminococcaceae sp., Faecalibacterium sp., Bacteroides thetaiotaomicron, Alistipes shahii, Holdemania sp., Streptococcus mutans, Dorea formicigenerans, Akkermansia sp., Enterococcus hirae, Alistipes sp., Bifidobacterium longum, Collinsella aerofaciens, Enterococcus faecium, Bacillus Calmette-Guerin, Parabacteroides goldsteinii, and Enterococcus gallinarum. In some embodiments, the PD-L1 inhibitor is atezolizumab, avelumab, or durvalumab. In some embodiments of the methods, compositions, and kits provided herein, the anticancer therapy includes the administration of a CTLA-4 inhibitor and one or more bacterial strains of species selected from the group consisting of Bacteroides sp., Burkholderia sp., Bifidobacterium sp., Ruminococcaceae sp., Faecalibacterium sp., Bacteroides thetaiotaomicron, Alistipes shahii, Holdemania sp., Streptococcus mutans, Dorea formicigenerans, Akkermansia sp., Enterococcus hirae, Alistipes sp., Bifidobacterium longum, Collinsella aerofaciens, Enterococcus faecium, Bacillus Calmette-Guerin, Parabacteroides goldsteinii, and Enterococcus gallinarum. In some embodiments, the CTLA-4 inhibitor ipilimumab or tremelimumab. In some embodiments of the methods, compositions, and kits provided herein, the anticancer therapy includes the administration of multiple immune checkpoint inhibitors (e.g., a PD-L1 inhibitor, and a CTLA-4 inhibitor) and one or more bacterial strains of species selected from the group consisting of Bacteroides sp., Burkholderia sp., Bifidobacterium sp., Ruminococcaceae sp., Faecalibacterium sp., Bacteroides thetaiotaomicron, Alistipes shahii, Holdemania sp., Streptococcus mutans, Dorea formicigenerans, Akkermansia sp., Enterococcus hirae, Alistipes sp., Bifidobacterium longum, Collinsella aerofaciens, Enterococcus faecium, Bacillus Calmette-Guerin, Parabacteroides goldsteinii, and Enterococcus gallinarum.

It should further be noted that certain bacterial strains are associated with enhancing the efficacy of a particular immune checkpoint inhibitor, as described in more detail above. As a non-limiting example, Dorea formicigenerans is associated with enhanced efficacy in combination with the PD-1 inhibitor; See, e.g., Frankel et al. Neoplasia (2017) 19: 848-855, and Frankel. et al. “Metagenomic shotgun sequencing to identify specific human gut microbes associated with immune checkpoint therapy efficacy in melanoma patients” (Abstract 9516 and Poster Board 124; ASCO 2017) A. E. Frankel, T. W. Froehlich, J. Kim, L. A. Coughlin, Y. Xie, E. P. Frenkel, A. Y). Thus, in some embodiments of the methods, compositions, and kits provided herein, the anticancer therapy includes the administration of a PD-1 inhibitor and one or more bacterial strains of species Dorea formicigenerans.

In addition, Akkermansia species, Enterococcus hirae, and Alistipes spp. are also associated with an increased efficacy with PD-1 inhibition; See, Routy et al. Science (2018) 359(6371): 91-97. Furthermore, Faecalibacterium spp and Ruminococcaceae were also associated with enhanced treatment with a PD-1 inhibitor; See Gopalakrishnan et al. Science (2017) 259(6371): 97-103. Furthermore, Bifidobacterium longum, Collinsella aerofaciens, and Enterococcus faecium are associated with enhanced efficacy in anti-PD1 and anti-PD-L1 efficacy; See Matson et al. Science (2018), 359; 104-108. Thus, in some embodiments of the methods, compositions, and kits provided herein, the anticancer therapy includes the administration of a PD-1 inhibitor or a PD-L1 inhibitor and one or more bacterial strains of species selected from the group consisting of Dorea formicigenerans, Akkermansia sp., Enterococcus hirae, Alistipes sp., Faecalibacterium spp, Ruminococcaceae, Bifidobacterium longum, Collinsella aerofaciens, and Enterococcus faecium. In some embodiments, the PD-1 inhibitor or PD-L1 inhibitor is nivolumab, pembrolizumab, atezolizumab, avelumab, or durvalumab.

Certain Bifidobacterium species are associated with enhanced efficacy of an anticancer therapy involving administration of a PD-L1 inhibitor; See, e.g., Sivan et al. Science (2015) 350(6264)). Thus, in some embodiments of the methods, compositions and kits provided herein, the anticancer therapy includes the administration of a PD-L1 inhibitor and one or more bacterial strains of species of Bifidobacterium. In some embodiments, the PD-L1 inhibitor is atezolizumab, avelumab, or durvalumab.

In addition, enhanced efficacy of treatment with the CTLA-4 inhibitor ipilimumab has also been associated with the presence of Faecalibacterium and other Firmicutes; See, Chaput et al. Ann. Oncol. (2017) 28(6): 1368-1379. Bacteroides species (e.g., B. fragilis, B. thetaiotaomicron) and Burkholderiales species are associated with enhanced efficacy of an anticancer therapy involving administration of a CTLA-4 inhibitor; See, e.g., Vétizou et al. Science (2015) 350(6264): 1079-1084; Pitt et al. Oncoimmunology (2017) 6(1)). Thus, in some embodiments of the methods, compositions and kits provided herein, the anticancer therapy includes the administration of a CTLA-4 inhibitor and one or more bacterial strains of species selected from the group consisting of Faecalibacterium spp, Bacteroides species B. fragilis, B. thetaiotaomicron and Burkholderiales. In some embodiments, the CTLA-4 inhibitor is ipilimumab or tremelimumab.

Additionally, it was found that subjects that responded positively to treatment with the PD-1 inhibitor nivolumab in combination with the CTLA-4 inhibitor ipilimumab had microbiomes enriched with Bacteroides thetaiotaomicron, Alistipes shahii, Holdemania, Faecalibacterium and Firmicutes, such as Ruminococcaceae, and Streptococcus mutans; See, e.g., Frankel et al. Neoplasia (2017) 19: 848-855, and Frankel. et al. “Metagenomic shotgun sequencing to identify specific human gut microbes associated with immune checkpoint therapy efficacy in melanoma patients.” (Abstract 9516 and Poster Board 124; ASCO 2017) A. E. Frankel, T. W. Froehlich, J. Kim, L. A. Coughlin, Y. Xie, E. P. Frenkel, A. Y). Thus, in some embodiments of the methods, compositions, and kits provided herein, the anticancer therapy includes the administration of a PD-1 inhibitor and a CTLA-4 inhibitor, and one or more bacterial strains of species selected from the group consisting of Bacteroides thetaiotaomicron, Alistipes shahii, Holdemania, Faecalibacterium and Firmicutes, such as Ruminococcaceae, and Streptococcus mutans. In some embodiments, the PD-1 inhibitor is nivolumab or pembrolizumab, and the CTLA-4 inhibitor is ipilimumab or tremelimumab.

### Suppressing Agents

Aspects of the present disclosure provide methods, compositions, and kits for use in anticancer therapy comprising a suppressing agent. The term “suppressing agent,” as used herein, refers to an agent that suppresses, or is capable of suppressing, one or more adverse events, e.g., toxicities, caused by the anticancer therapy and/or suppresses the immune response. In some embodiments, the suppressing agent suppresses one or more adverse events caused by the anticancer therapy. The method of any one of the preceding claims, wherein the suppressing agent is an agent that suppresses the immune response. The suppressing agent may generally suppress the immune response in the subject or more specifically suppress a particular aspect of an immune response.

In some embodiments, administration of a suppressing agent involves administration of one or more (e.g., 2, 3, 4, 5, or more) suppressing agents. As described herein, in some embodiments, the method involves administering a suppressing agent once, as a single administration. In some embodiments, the method involves administering a suppressing agent multiple times. In some embodiments, the method may involve multiple administration of the same suppressing agent or multiple administration of different suppressing agents.

Also within the scope of the present disclosure are combinations of suppressing agents. For example, one component of a combination of suppressing agent may enhance the efficacy (e.g., suppressing effects) of another suppressing agent of the combination. As will be appreciated by one of skill in the art, the suppressing agents of the combination of suppressing agents may be administered to the subject concomitantly. In some embodiments, the combination of suppressing agents is administered to the subject in more than one composition. In some embodiments, the suppressing agents or the combination of suppressing agents are administered simultaneously to the subject. In some embodiments, the combination of suppressing agent is administered to the subject in the same composition. In some embodiments, the anticancer therapies of the combination of suppressing agents are administered sequentially to the subject.

In some embodiments, the suppressing agent induces a desired immune response. For example, in some embodiments, the suppressing agent induces the proliferation and/or accumulation of regulatory T cells, also referred to as “Tregs” or “Treg cells”. The abundance of regulatory T cells can be assessed by any method known in the art, for example by detecting a cellular marker indicative of regulatory T cells (e.g., FoxP3), assessing a direct or indirect activity of regulatory T cells, and/or by measuring the production of one or more cytokines produced by regulatory T cells (e.g. IL-10).

In some embodiments, the suppressing agent is a suppressing live bacterial product. In some embodiments, the suppressing live bacterial product induces regulatory T cells. As will be appreciated by one of skill in the art, one or more bacterial strains may be selected and combined in a suppressing live bacterial product. As described herein, a suppressing live bacterial product may comprise at least 1, 2, 3, 4, 5, 6, 7, 8, 9, 10, 11, 12, 13, 14, 15, 16, 17, 18, 19, 20, 21, 22, 23, 24, 25, 26, 27, 28 29, 30 or more bacterial strains.

Examples of live bacterial productions capable of suppressing an adverse event and/or inducing regulatory T cells are known in the art. See, for example PCT Publication Nos. WO 2011/152566, WO 2013/080561, WO 2016/209806, and WO2017/218680, and Atarashi et al., Nature (2013) 500: 232-236 and Geva-Zatorsky et al. Cell (2017) 168, 928, which are all herein incorporated by reference in their entirety.

In some embodiments, the suppressing live bacterial product comprises one or more bacterial strains belonging to Clostridium cluster XIVa. In some embodiments, the suppressing live bacterial product comprises one or more bacterial strains belonging to Clostridium cluster IV. In some embodiments, the suppressing live bacterial product comprises one or more bacterial strains belonging to Clostridium cluster XIVa and/or Clostridium cluster IV. In some embodiments, the suppressing live bacterial product comprises one or more bacterial strains belonging to Clostridium cluster XVIII. In some embodiments, the suppressing live bacterial product comprises one or more bacterial strains belonging to Clostridium cluster XIVa and/or Clostridium cluster IV, and/or Clostridium cluster XVIII.

In some embodiments, the suppressing live bacterial product comprises at least one bacteria selected from Clostridium saccharogumia, Clostridium ramosum JCM1298, Clostridium ramosum, Flavonifractor plautii, Pseudoflavonifractor capillosus ATCC 29799, Clostridium hathewayi, Clostridium saccharolyticum WM1, Bacteroides sp. MANG, Clostridium saccharolyticum, Clostridium scindens, Lachnospiraceae bacterium 5_1_57FAA, Lachnospiraceae bacterium 6_1_63FAA, Clostridium sp. 14616, Clostridium bolteae ATCC BAA-613, cf. Clostridium sp. MLG055, Erysipelotrichaceae bacterium 2_2_44A, Clostridium indolis, Anaerostipes caccae, Clostridium bolteae, Lachnospiraceae bacterium DJF_VP30, Lachnospiraceae bacterium 3_1_57FAA_CT1, Anaerotruncus colihominis, Anaerotruncus colihominis DSM 17241, Ruminococcus sp. ID8, Lachnospiraceae bacterium 2_1_46FAA, Clostridium lavalense, Clostridium asparagiforme DSM 15981, Clostridium symbiosum, Clostridium symbiosum WAL-14163, Eubacterium contortum, Clostridium sp. D5, Oscillospiraceae bacterium NML 061048, Oscillibacter valericigenes, Lachnospiraceae bacterium A4, Clostridium sp. 316002/08, Clostridiales bacterium 1_7_47FAA, Blautia coccoides, and Anaerostipes caccae DSM 14662. See also Atarashi et al., Nature (2013) 500:232-236 and PCT publication WO 2013/080561.

In some embodiments, the suppressing live bacterial product is VE-202. In some embodiments, the suppressing live bacterial product contains Clostridium saccharogumia, Flavonifractor plautii, Clostridium hathewayi, Blautia coccoides, Clostridium bolteae ATCC BAA-613, cf. Clostridium sp. MLG055, Clostridium indolis, Anaerotruncus colihominis, Ruminococcus sp. ID8, Clostridium asparagiforme DSM 15981, Clostridium symbiosum, Clostridium ramosum, Eubacterium contortum, Lachnospiraceae bacterium 5_1_57FAA, Lachnospiraceae bacterium 3_1_57FAA_CT1, Clostridiales bacterium 1_7_47FAA, and Lachnospiraceae bacterium A4. It should be appreciated that subsets of the VE-202 bacteria can also induce Treg cells. Examples of subsets of VE202 that induce Treg cells are found for instance in Atarashi et al., Nature (2013) 500: 232-236 and corresponding Supplemental Information and PCT publication WO 2013/080561.

In some embodiments, the suppressing live bacterial product is any of the bacterial compositions as described in PCT publication WO 2016/209806. In some embodiments, the suppressing live bacterial product contains one or more bacteria selected from Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter Iwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii, and Bifidobacterium breve. In some embodiments, the suppressing live bacterial product does not include a bacterial strain of the Clostridia class.

In some embodiments, the suppressing live bacterial product is any of the bacterial compositions as described in PCT publication WO 2017/218680. In some embodiments, the suppressing live bacterial product contains one or more bacteria selected from the following groups:

1) Clostridium bolteae,

2) Anaerotruncus colihominis,

3) Eubacterium fissicatena, Dracourtella massiliensis, Ruminococcus torques, or Sellimonas intestinalis,

4) Clostridium symbiosum,

5) Blautia producta,

6) Dorea longicatena,

7) Clostridium innocuum, and

8) Flavinofractor plautii, Clostridium orbiscindens, or Subdolinogranulum sp.

In some embodiments, the suppressing live bacterial product contains one or more bacterial strains of each of the eight recited groups. It should be appreciated that certain subsets of the eight bacterial groups recited above also induce Treg cells and that such subsets, in some embodiments, can be used in the compositions, methods, and kits of the disclosure.

In some embodiments, administration of the suppressing live bacterial product results in the repopulation of the microbiota of the subject with bacterial strains from the suppressing live bacterial product.

Also within the scope of suppressing agents are immunomodulatory agents that specifically suppress one or more aspect of an immune response. The suppressing agent may generally suppress the immune response in the subject, or, more specifically, suppress a particular aspect of an immune response.

In some embodiments, the suppressing agent is a steroid, such as a corticosteroid. In some embodiments, the suppressing agent is dexamethasone, hydrocortisone, prednisone, prednisolone, or methylprednisolone.

In some embodiments, the suppressing agent inhibits a pro-inflammatory molecule (e.g., a proinflammatory cytokine). In some embodiments, the suppressing agent targets TNFα. In some embodiments, the suppressing agent is an antibody. In some embodiments, the suppressing agent is infliximab, adalimumab, certolizumb, golimumab, or etanercept.

In some embodiments, the disclosure provides methods, compositions and kits comprising multiple suppressing agents. Thus, for instance, in some embodiments, the disclosure provides methods, compositions and kits comprising the administration of a suppressing live bacterial product and a steroid. In addition, for example, in some embodiments, the disclosure provides methods, compositions and kits comprising the administration of multiple suppressing live bacterial products.

### Live Bacterial Products

It should be appreciated that the anticancer live bacterial products and/or the suppressing live bacterial products of the methods, compositions, and kits provided herein may be bacterial products (e.g., bacterial cocktails) with certain desired characteristics. The live bacterial products may be also referred to herein as bacterial cocktails, live bacterial consortia, or bacterial consortia, comprise one or more bacterial strains. Bacterial strains may be selected and combined, as described herein, to compose a live bacterial product having one or more desired characteristics.

It should be appreciated that closely related bacterial strains (e.g., as defined by 16S rDNA sequences) have similar or the same biological properties. In some embodiments, bacterial strains provided herein can be replaced with bacterial strains with similar properties. Thus, in one aspect, the disclosure provides bacterial strains with 16S rDNA sequences that have homology to a nucleic acid sequence of any one of the sequences of the bacterial strains or species described herein. In some embodiments, the bacterial strain has at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or up to 100% homology relative to any one of the strains or bacterial species described herein over a specified region or over the entire sequence. It would be appreciated by one of skill in the art that the term “homology” or “percent homology,” in the context of two or more nucleic acid sequences or amino acid sequences, refers to a measure of similarity between two or more sequences or portion(s) thereof. The homology may exist over a region of a sequence that is at least about 50 nucleotides in length, or more preferably over a region that is 100 to 500 or 1000 or more nucleotides in length. In some embodiments, the homology exists over the length the 16S rRNA or 16S rDNA sequence, or a portion thereof.

Additionally, or alternatively, two or more sequences may be assessed for the identity between the sequences. The terms “identical” or percent “identity” in the context of two or more nucleic acids or amino acid sequences, refer to two or more sequences or subsequences that are the same. In some embodiments, the bacterial strain has at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or up to 100% sequence identity with any one of the strains or bacterial species described herein over a specified region or over the entire sequence. Two sequences are “substantially identical” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the following sequence comparison algorithms or by manual alignment and visual inspection. Optionally, the identity exists over a region that is at least about 50 nucleotides in length, or more preferably over a region that is 100 to 500 or 1000 or more nucleotides in length. In some embodiments, the identity exists over the length the 16S rRNA or 16S rDNA sequence.

Additionally, or alternatively, two or more sequences may be assessed for the alignment between the sequences. The terms “alignment” or percent “alignment” in the context of two or more nucleic acids or amino acid sequences, refer to two or more sequences or subsequences that are the same. Two sequences are “substantially aligned” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the following sequence comparison algorithms or by manual alignment and visual inspection. Optionally, the alignment exists over a region that is at least about 50 nucleotides in length, or more preferably over a region that is 100 to 500 or 1000 or more nucleotides in length. In some embodiments, the identity exists over the length the 16S rRNA or 16S rDNA sequence.

For sequence comparison, typically one sequence acts as a reference sequence, to which test sequences are compared. Methods of alignment of sequences for comparison are well known in the art. See, e.g., by the local homology algorithm of Smith and Waterman (1970) Adv. Appl. Math. 2:482c, by the homology alignment algorithm of Needleman and Wunsch, J. Mol. Biol. 48:443, 1970, by the search for similarity method of Pearson and Lipman. Proc. Natl. Acad. Sci. USA 85:2444, 1988, by computerized implementations of these algorithms (GAP, BESTFIT, FASTA, and TFASTA in the Wisconsin Genetics Software Package, Genetics Computer Group. Madison. Wis.), or by manual alignment and visual inspection (see. e.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

In one aspect, the disclosure provides live bacterial products (e.g., anticancer and/or suppressing live bacterial products) that contain multiple purified bacterial strains.

In some embodiments, one or more of the bacterial strains are human-derived bacteria, meaning the one or more bacterial strains were obtained from or identified from a human or a sample therefrom (e.g., a human donor). In some embodiments of the compositions provided herein, all of the bacterial strains are human-derived bacteria. In some embodiments of the compositions provided herein, the bacterial strains are derived from more than one human donor.

The bacterial strains used in the live bacterial products provided herein generally are isolated from the microbiome of healthy individuals. In some embodiments, the live bacterial products include strains originating from a single individual. In some embodiments, the live bacterial products include strains originating from multiple individuals. In some embodiments, the bacterial strains are obtained from multiple individuals, isolated and grown up individually. The bacterial compositions that are grown up individually may subsequently be combined to provide the compositions of the disclosure. It should be appreciated that the origin of the bacterial strains of the live bacterial products provided herein is not limited to the human microbiome from a healthy individual. In some embodiments, the bacterial strains originate from a human with a microbiome in dysbiosis. In some embodiments, the bacterial strains originate from non-human animals or the environment (e.g., soil or surface water). In some embodiments, the combinations of bacterial strains provided herein originate from multiple sources (e.g., human and non-human animals).

In some embodiments, the live bacterial product includes one or more anaerobic bacteria. In some embodiments, the live bacterial product includes only anaerobic bacteria. In some embodiments, the live bacterial product includes one or more facultative anaerobic bacteria. In some embodiments, the live bacterial product includes only facultative anaerobic bacteria. In some embodiments, the live bacterial product includes one or more obligate anaerobic bacteria. In some embodiments, the live bacterial product includes only obligate anaerobic bacteria.

In some embodiments, one or more of the bacterial strains of the live bacterial products does not have an antibiotic resistance gene. In some embodiments, the bacterial strains do not have an antibiotic resistance gene that renders the bacterial strain resistant to vancomycin.

In some embodiments, the live bacterial product does not include bacterial strains that are resistant to one or more antibiotics. It should be appreciated that it may be desirable to have a mechanism to remove the bacterial compositions provided herein from the body after administration. One such mechanism is to remove the bacterial compositions by antibiotic treatment. Thus, in some embodiments, the live bacterial product does not include bacterial strains that are resistant to one or more antibiotics. In some embodiments, the live bacterial products do not include bacterial strains that are resistant to one or more antibiotics selected from the group consisting of penicillin, benzylpenicillin, ampicillin, sulbactam, amoxicillin, clavulanate, tazobactam, piperacillin, cefmetazole, vancomycin, imipenem, meropenem, metronidazole, and clindamycin. In some embodiments, the live bacterial products do not include bacterial strains that are resistant to vancomycin.

As used herein, an “antibiotic that is efficacious in a human” refers to an antibiotic that has been used to successfully treat bacterial infections in a human. In some embodiments, the live bacterial products include bacterial strains that are susceptible to at least four antibiotics that are efficacious in humans. In some embodiments, the live bacterial products include bacterial strains that are susceptible to at least three antibiotics that are efficacious in humans. In some embodiments, the live bacterial products include bacterial strains that are susceptible to at least two antibiotics that are efficacious in humans. In some embodiments, the live bacterial products include bacterial strains that are susceptible to at least one antibiotic that is efficacious in humans. In some embodiments, the live bacterial products include only bacterial strains that are susceptible to at least four antibiotics that are efficacious in humans. In some embodiments, the live bacterial products include only bacterial strains that are susceptible to at least three antibiotics that are efficacious in humans. In some embodiments, the live bacterial products include only bacterial strains that are susceptible to at least two antibiotics that are efficacious in humans. In some embodiments, the live bacterial products include bacterial strains that are susceptible to at least one antibiotic that is efficacious in humans.

In some embodiments, one or more of the bacterial strains of the live bacterial product is a spore-former. In some embodiments, one or more of the bacterial strains is in spore form. In some embodiments, one or more of the bacterial strains is a non-spore former.

In some embodiments, the live bacterial products described herein comprise spore forming and non-spore forming bacterial strains. In some embodiments, the live bacterial products described herein comprise spore-forming bacterial strains. In some embodiments, the live bacterial products described herein comprise only spore-forming bacterial strains. In some embodiments, the live bacterial products described herein comprise only non-spore forming bacterial strains. It should be appreciated that the spore-forming bacteria can be in spore form (i.e., as spores) or in vegetative form (i.e., as vegetative cells). In spore form, bacteria are generally more resistant to environmental conditions, such as heat, acid, radiation, oxygen, chemicals, and antibiotics. In contrast, in the vegetative state or actively growing state, bacteria are more susceptible to such environmental conditions, compared to in the spore form. In general, bacterial spores are able to germinate from the spore form into a vegetative/actively growing state, under appropriate conditions. For instance, bacteria in spore format may germinate when they are introduced in the intestine.

In some embodiments, at least one (e.g., 1, 2, 3, 4, 5, or more) of the bacterial strains in the live bacterial products provided herein is a spore former. In some embodiments, at least one (e.g., 1, 2, 3, 4, 5, or more) of the bacterial strains is in spore form. In some embodiments, at least one (e.g., 1, 2, 3, 4, 5, or more) of the bacterial strains is a non-spore former. In some embodiments, at least one (e.g., 1, 2, 3, 4, 5, or more) of the bacterial strains is in vegetative form. As discussed above, spore forming bacteria can also be in vegetative form. In some embodiments, at least one (e.g., 1, 2, 3, 4, 5, or more) of the bacterial strains is in spore form and at least one (e.g., 1, 2, 3, 4, 5, or more) of the bacterial strains in the composition is in vegetative form. In some embodiments, at least one bacterial strain that is considered able to form spores (i.e., a spore-former) is present in vegetative form. In some embodiments, at least one bacterial strain that is considered able to form spores is present in the live bacterial product both in spore form and in vegetative form.

It is envisioned that the bacterial strains of the live bacterial products provided herein are alive and will be alive when they reach the target area (e.g., the intestines). Bacterial spores are considered to be alive in this regard. In some embodiments, bacteria that are administered as spores may germinate in the target area (e.g., the intestines). It should further be appreciated that not all of the bacteria are alive, and the compositions can include a percentage (e.g., by weight) that is not alive. In addition, in some embodiments, the compositions include bacterial strains that are not alive when administered or at the time when the composition reaches the target area (e.g., the intestines). It is envisioned that non-living bacteria may still be useful by providing some nutrients and metabolites for the other bacterial strains in the composition.

In any of the live bacterial products provided herein, in some embodiments, the bacterial strains are purified. In any of the live bacterial products provided herein, in some embodiments, the bacterial strains are isolated. Any of the bacterial strains described herein may be isolated and/or purified, for example, from a source such as a culture or a microbiota sample (e.g., fecal matter). The bacterial strains used in the compositions provided herein generally are isolated from the microbiome of healthy individuals. However, bacterial strains can also be isolated from individuals that are considered not to be healthy. In some embodiments, the compositions include strains originating from multiple individuals. As used herein, the term “isolated” bacteria refers to bacteria that have been separated from one or more undesired component, such as another bacterium or bacterial strain, one or more component of a growth medium, and/or one or more component of a sample, such as a fecal sample. In some embodiments, the bacteria are substantially isolated from a source such that other components of the source are not detected. As also used herein, the term “purified” refers to a bacterial strain or composition comprising such that has been separated from one or more components, such as contaminants. In some embodiments, the bacterial strain is substantially free of contaminants. In some embodiments, one or more bacterial strains of a live bacterial product may be independently purified from one or more other bacteria produced and/or present in a culture or a sample containing the bacterial strain. In some embodiments, a bacterial strain is isolated or purified from a sample and then cultured under the appropriate conditions for bacterial replication, e.g., under anaerobic culture conditions. The bacteria that is grown under appropriate conditions for bacterial replication can subsequently be isolated/purified from the culture in which it is grown.

### Adverse Events

Aspects of the present disclosure provide methods, compositions, and kits for suppressing adverse events that are caused by administration of an anticancer therapy (e.g., an anticancer agent). The term “adverse event” may be used interchangeably with the term “side effect” or toxicity” and refers to any undesired effect that is caused by administration of the anticancer therapy. An adverse event is considered to be caused by the anticancer therapy if the adverse event occurs subsequent to the initiation of the anticancer therapy. In general, the adverse event may be directly or indirectly caused by the anticancer therapy. In some embodiments, the adverse event is used as an indicator that the anticancer therapy is effective.

Examples of adverse events that may be caused by anticancer therapy include, without limitation, undesired immune responses, colitis, inflammation, dermatological toxicity, diarrhea, nausea, fatigue, hepatotoxicity, hypophysitis, eosinophilia, and autoimmune thyroid disease. Additional adverse events caused by the anticancer therapy will be evident to one of skill in the art.

Adverse events associated with checkpoint inhibitor therapy are summarized for instance in Postow et al., Up to Date June 2017, Toxicities associated with checkpoint inhibitor immunotherapy, authors Postow M and Wolchok J, editors Atkins M and Ross M., Yang et al., Recognizing and managing toxicities in cancer immunotherapy, Tumor Biology March (2017): 1-13; and Linardou et al., Toxicity management of immunotherapy for patients with metastatic melanoma”. Ann Transl. Med (2016) 4 (14) 22, which are all incorporated herein by reference in their entirety.

In some embodiments, the adverse event is immune-checkpoint inhibitor induced colitis (See e.g., Prieux-Clotz et al., Target Oncology 2017, 12, 301-308).

As will be appreciated by one of skill in the art, an adverse event may occur at any time after administration of an anticancer therapy. In some embodiments, the adverse event caused by the anticancer agent occurs immediately (e.g., less than 1 hour) after initiation of the anticancer therapy. In some embodiments, the adverse event caused by the anticancer agent occurs within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more after initiation of the anticancer therapy.

In some embodiments, the adverse event occurs after termination of the anticancer therapy. In some embodiments, the adverse event caused by the anticancer agent occurs within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more after termination of the anticancer therapy.

In some embodiments, the adverse event occurs immediately after a round of the anticancer therapy. In some embodiments, the adverse event caused by the anticancer agent occurs within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more after one round of the anticancer therapy.

The methods described herein involve administering a suppressing agent to reduce or eliminate an adverse event that is caused by the anticancer therapy. Also within the scope of the present disclosure are methods for determining whether an adverse event has occurred or is occurring in the subject. In some embodiments, if the occurrence of an adverse event is determined, one or more suppressing agents, as described herein, are administered to the subject.

In some embodiments, the suppressing agent is administered prior to the administration of the anticancer therapy. It should be appreciated that the presence of certain agents or bacterial strains in a subject are associated with the suppression of adverse events (e.g., the presence of Treg inducing bacteria). Thus, in some embodiments, it is beneficial to introduce agents or bacterial strains that are associated with the suppression of adverse events, prior to the initiation of the anticancer therapy. In some embodiments, one or more suppressing agents (e.g., suppressing live bacterial cocktails) are administered and the level of the suppression agent in the subject is determined. If the level of the suppression agent is below a level that is associated with suppression of adverse events, additional administrations of the suppressing agent may be provided to result in a level of the suppressing agent associated with protection against adverse events. In some embodiments, anticancer therapy is initiated only if the suppressing agents are present in levels sufficient to protect against adverse events.

In some embodiments, the suppressing agent is administered at the same time as the anticancer therapy. It should be appreciated that, in some embodiments, simultaneous administration of the suppressing agent and the anticancer therapy results in the suppression of adverse events of the anticancer therapy.

Determining the occurrence of adverse events can be done through a variety of methods known in the art. Also described herein are diagnostic methods (e.g., companion diagnostics) for use in determining whether an adverse event has occurred, or is occurring in a subject, and, if so, that the subject should receive a suppressing agent. Such methods can be used for diagnosing an adverse event, monitoring the progress of the adverse event, assessing the efficacy of the suppressing agent in reducing the adverse event, and/or identifying patients suitable for a particular treatment.

In some embodiments, the occurrence of an adverse event may be determined based on the presence or severity of one or more symptoms associated with an adverse event. In general, the presence or severity of a symptom may be scored and given a value that may be compared to a score for the symptom in the subject at a different time (e.g., prior to or after administration of any of the therapies described herein).

In some embodiments, the occurrence of an adverse event may be determined based on the level of a marker (e.g., a biomarker) in a sample obtained from a subject. In some embodiments, the methods involve analyzing the presence and/or level of a marker in one or more samples from a subject. In some embodiments, the sample is a biological sample, such as a tissue sample (e.g., biopsy), a blood or plasma sample, a sputum sample, a fecal sample, or a urine sample.

In some embodiments, the level of the marker is determined by analyzing the expression of the marker (e.g., protein or nucleic acid level) and/or the cell type in which the marker is expressed. Any method known in the art may be used to analyze the expression of the marker and/or cell type in which the marker is expressed. As will be appreciated by one of skill in the art, the determination of whether an adverse event has occurred may depend on the nature of the adverse event.

In some embodiments, the adverse event is an adverse event that occurs in the gut.

In some embodiments, the occurrence of an adverse event is determined by assessing an aspect of the immune response. In some embodiments, the presence or quantity of a specific cell type or subtype is assessed in a sample from the subject to assess if an adverse event has or will occur. In some embodiments, the presence of quantity of white blood cells is assessed in a sample from the subject. In some embodiments, the presence of quantity of T cells or B cells is assessed in a sample from the subject. In some embodiments, the presence of quantity of eosinophils is assessed in a sample from the subject. For example, it has been demonstrated that eosinophilia may be associated with the occurrence of an adverse event caused by anticancer therapy. See, Schindler et al. J. Clin. Oncol. (2014): 32:5s: Abstract 9096. It should be appreciated that assessing aspects of the immune response can also be used to assess the likelihood that an adverse event can occur. Thus, for instance, the level of Treg cells can be determined, which, in some embodiments, is a marker for the likelihood an adverse event will occur. In some embodiments, a higher level of Treg cells is associated with a higher level of protection against adverse events. It should be appreciated that if a level of a suppressing agent is measured (e.g., the level of Treg cells), and found to be insufficient to provide protection against adverse events, additional doses of suppressing agent may be provided prior to starting or resuming anticancer therapy.

Alternatively, or in addition, the presence or quantity of a cytokine is assessed in a sample from the subject, which may be indicative of whether an adverse event has occurred. In some embodiments, the presence or quantity of an inflammatory cytokine is assessed in a sample from the subject. In some embodiments, the presence or quantity of any one or more of IL-17, IL-1, IL-12, IL-6, IL18, TNF, IFNγ, and GM-CSF are assessed in a sample from the subject. For example, it has been demonstrated that increased levels of IL-17 in a subject's serum may be associated with colitis; See, Calahan et al. J. Clin. Oncol. (2011): 29s: Abstract 2505. In some embodiments, the presence or level of an immunosuppressive cytokine is assessed in a sample from the subject. In some embodiments, the presence or quantity of any one or more of IL-10, TGF-β, IL-33, IL4, IL-13, IL-37, and IL-35 are assessed in a sample from the subject.

Any one or more markers indicating the activity level or health of a specific cell type or organ may be assessed to determine whether an adverse event has occurred in the subject. For example, plasma levels of an enzyme, hormone, or other molecule may be indicative of the function of an organ, such as the liver, kidneys, pituitary gland, or thyroid.

In some embodiments, determining whether an adverse event has occurred may involve evaluating the appearance of the subject's skin overall or a specific region of the skin. In some embodiment, the adverse event is a rash or mucosal irritation. For example, the overall coloring of the skin may be an indication of the function or dysfunction of an organ, e.g., jaundice. In some embodiments, topical examination of a subject's skin or a region of the skin may indicate whether the subject is experiencing dermatological toxicity.

In some embodiments, determining whether an adverse event has occurred may involve evaluating a stool sample from the subject. In some embodiments, the stool sample may be evaluated to assess the presence or quantity of white blood cells and/or red blood cells in the stool, which may be indicative of the conditions in the small intestine or colon of the subject. In some embodiments, the adverse event can be assessed by measuring an inflammation marker in the blood (e.g., calprotectin). In some embodiments, the stool sample may be evaluated to assess the presence or quantity of bacteria, viruses, and/or parasites, including specific species thereof. In some embodiments, additional characteristics of the stool are evaluated as an indication of whether an adverse event has occurred, for example, using the Bristol stool scale. See also PCT publication WO2017/091694, which describes the use of bacterial markers for the identifying subject at risk for checkpoint blockade therapy associated colitis.

As described herein, in some embodiments, the methods involve determining the presence and/or level of a marker in one or more samples from a subject. In some embodiments, the level of the marker in a sample obtained from a subject can then be compared with a reference sample or a control sample to determine a value indicating the amount of the marker in the sample. In some embodiments, a value for a marker is obtained by comparing the level of a marker in a sample to the level of another marker (e.g., an internal control or internal standard) in the sample. The value of the marker can be compared to a reference value to determine whether the subject has or is at risk for the adverse event. In some embodiments, the level of the marker is compared to a predetermined threshold for the marker, a deviation from which may indicate the subject has experienced an adverse event. In some embodiments, if the level or value of the marker is higher than a reference level or value, the subject can be identified as having or at risk for an adverse event, as described herein. In some embodiments, if the level or value of the marker is lower than a reference level or value, the subject can be identified as having or at risk for an adverse event, as described herein.

In some embodiments, the level of the marker in a sample from a subject is compared to the level of the marker in another sample obtained from the same subject, for example, a sample obtained from the subject at a different time. In some embodiments, the level of the marker in a sample from a subject is compared to the level of the marker in a sample obtained from the subject at an earlier time, such as prior to administration of any of the therapies described herein. In some embodiments, the level of the marker in a sample from a subject is compared to the level of the marker in a sample obtained from the subject prior to administration of a suppressing agent, for example to determine the efficacy of the suppressing agent. In some embodiments, the level of the marker in a sample from a subject is compared to the level of the marker in a sample obtained from the subject at a later time, such as after administration of any of the therapies described herein. In some embodiments, the level of the marker in a sample from a subject is compared to the level of the marker in a sample obtained from the subject at a later time, such as after administration of any of the anticancer therapies described herein.

In some embodiments, if the level or value of the marker is higher in a sample as compared to a level or value of the marker in a sample from the subject obtained prior to administration of a composition described herein, the subject is administered a suppressing agent as described herein. In some embodiments, the level or value of the marker in a sample is enhanced at least 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or at least 200% as compared the level of value of the marker in a sample prior to administration of the suppressing agent as described herein.

In some embodiments, if the level or value of the marker is not increased (e.g., equal to or lower) in a sample as compared to the level or value of the marker in a sample from the subject obtained prior to administration of a suppressing agent described herein, the administration of the suppressing agent is reanalyzed after administration of one or more additional doses of the suppressing agent. In some embodiments, the level or value of the marker in a sample is reduced at least 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or at least 200% as compared the level of value of the marker in a sample prior to administration of a suppressing agent as described herein.

### Treatment of Cancer

Aspects of the present disclosure include methods, compositions and kits for the treatment of diseases (e.g., cancer) in a subject. In some embodiments, the subject has cancer or is at risk of developing cancer. In some embodiments, the subject is undergoing or has undergone anticancer therapy.

Examples of cancers that can be treated according to the and methods provided herein, include without limitation, carcinoma, glioma, mesothelioma, melanoma, lymphoma, leukemia, adenocarcinoma, breast cancer, ovarian cancer, cervical cancer, glioblastoma, multiple myeloma, prostate cancer, Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, skin cancer, retinoblastomas, Hodgkin's lymphoma, non-Hodgkin's lymphoma, Kaposi's sarcoma, multicentric Castleman's disease, AIDS-associated primary effusion lymphoma, neuroectodermal tumors, or rhabdomyosarcoma. In some embodiments of the methods provided herein, the cancer is prostate cancer, bladder cancer, non-small cell lung cancer, urothelial carcinoma, melanoma, Merkel cell cancer, or renal cell carcinoma. In some embodiments, the cancer is melanoma, non-small cell lung cancer (NSCLC), Hodgkin's lymphoma, head and neck cancer, renal cell cancer, bladder cancer, or Merkel cell carcinoma.

In some embodiments, the cancer is melanoma and the anticancer therapy involves administering a CTLA-4 inhibitor (e.g., ipilimumab, tremelimumab). In some embodiments, the cancer is melanoma, NSCLC, Hodgkin's lymphoma, renal cancer, head and neck cancer and the anticancer therapy involves administering a PD-1 inhibitor (e.g., pembrolizumab, nivolumab). In some embodiments, the cancer is bladder cancer, NSCLC, or Merkel cell carcinoma and the anticancer therapy involves administering a PD-L1 inhibitor (e.g., atezolizumab, avelumab, durvalumab).

In one aspect, the disclosure provides methods, compositions. and kits for the treatment of cancer in a subject. In one aspect, the disclosure provides methods, compositions. and kits for suppressing adverse events associated with the treatment of cancer in a subject. As used herein, “subject,” “individual,” and “patient” are used interchangeably, and refer to a vertebrate, preferably a mammal such as a human. Mammals include, but are not limited to, human primates, non-human primates or murine, bovine, equine, canine or feline species. In some embodiments, the subject has or is at risk of having cancer. In some embodiments, the subject is a human cancer patient. In some embodiments, the subject has experienced an adverse event associated with an anticancer therapy.

Any of the anticancer agents and/or suppressing agents described herein may be administered to a subject in a therapeutically effective amount or a dose of a therapeutically effective amount to treat or prevent a disease (e.g., cancer) or to treat, suppress or prevent an adverse event. The terms “treat” or “treatment” refer to reducing or alleviating one or more of the symptoms associated with a disease (e.g., cancer). The terms “prevent” or “prevention” encompass prophylactic administration and may reduce the incidence or likelihood of the occurrence of the disease (e.g., cancer). For instance, in some embodiments, administration of the compositions provided herein result in a healthy microbiome that provides an anticancer effect in a subject. In some embodiments, administration of the compositions provided herein result in a healthy microbiome that suppresses one or more adverse effects caused by anticancer therapy in a subject.

In some embodiments, the therapeutically effective amount of any of the compositions described herein is an amount sufficient to treat the cancer and/or one or more adverse events (side effects) caused by the anticancer therapy. In some embodiments, the therapeutically effective amount of any of the compositions described herein enhances survival of the subject, suppresses an adverse event and/or treats the cancer. In some embodiments, the therapeutically effective amount of any of the compositions described herein is an amount sufficient to populate the intestine of the subject with bacteria of a composition. In some embodiments, populating the intestine of the subject with bacteria of a composition results in the suppression of side effects.

As used herein, the term “therapeutically effective amount” may be used interchangeably with the term “effective amount.” A therapeutically effective amount or an effective amount of a composition, such as a pharmaceutical composition, anticancer therapy, and/or a suppressing agent, is any amount that results in a desired response or outcome in a subject, such as those described herein, including but not limited to delay the manifestation, arrest the progression, relieve and/or alleviate at least one symptom of the disease that is treated using the methods described herein (e.g., cancer). A therapeutically effective amount of a suppressing agent, for example, may be any amount that results in a reduction or elimination of an adverse event caused by an anticancer therapy or one or more symptoms associated with an adverse event caused by an anticancer therapy. Note that when a combination of active ingredients is administered the effective amount of the combination may or may not include amounts of each ingredient that would have been effective if administered individually.

It should be appreciated that the term effective amount, in reference to a live bacterial product, may be expressed, for instance, in number of bacteria or colony forming units (CFUs) to be administered. It should further be appreciated that the bacteria can multiply once administered. Thus, administration of even a relatively small number of bacteria may have therapeutic effects.

Any of the methods described herein may be for the treatment of cancer in a subject. As used herein, methods of treating cancer involve relieving or alleviating at least one symptom associated with the cancer, or slowing or reversing the cancer progression. A method of treating cancer may, for example, eliminate or reduce a subject's tumor burden, reduce the number or replication of cancer cells, and/or prevent, delay or inhibit metastasis.

Any of the methods described herein may be for the suppression of an adverse effect caused by an anticancer therapy. As used herein, method of suppressing an adverse effect caused by an anticancer therapy involve reducing or eliminating an adverse event and/or relieving or alleviating at least one symptom associated with the adverse event. In some embodiments, the methods described herein suppress one or more adverse events caused by an anticancer therapy and thereby allow the subject to undergone additional anticancer therapy (e.g., one or more additional rounds of anticancer therapy). In some embodiments, the methods described herein suppress one or more immune responses, for example by inducing a regulatory T cell response. In some embodiments, the method described herein also repopulate the microbiota of the subject following anticancer therapy.

The efficacy of the therapeutic methods described herein using the anticancer therapy and suppressing agent(s) may be assessed by any method known in the art and would be evident to a skilled medical professional. For example, the efficacy of the therapy may be assessed by survival of the subject or cancer burden in the subject or tissue or sample thereof. In some embodiments, the efficacy of the therapy is assessed by quantifying the number of cells belonging to a particular population of cells (e.g. cancer cells, or cells of the immune system or subclasses thereof). In some embodiments, the efficacy of the therapy is assessed by evaluating the occurrence or severity of one or more adverse events in the subject.

In some embodiments, the subject is undergoing an anticancer therapy or has previously undergone an anticancer therapy.

In some embodiments, the subject is administered one or more suppressing agents that suppress an adverse event caused by the anticancer agent. In some embodiments, the suppressing agent is administered immediately after the onset of one or more adverse events. In some embodiments, the suppressing agent is administered within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months 6 months or more after the onset of adverse events.

In some embodiments, the suppressing agent is administered immediately after it has been determined that the subject is experiencing or has experienced an adverse event. In some embodiments, the suppressing agent is administered within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months 6 months or more after it has been determined that the subject is experiencing or has experienced an adverse event.

In some embodiments, the suppressing agent is administered prior to the onset of an adverse event. In some embodiments, the suppressing agent is administered prior to the determination that the subject is experiencing or has experienced an adverse event. In some embodiments, the suppressing agent is administered concurrently with the anticancer therapy. In some embodiments, the suppressing agent is administered within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more after the initiation of the anticancer therapy but prior to the onset or determination of an adverse event. In some embodiments, the suppressing agent is administered within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more after termination of the anticancer therapy but prior to the onset or determination of an adverse event.

In some embodiments, the suppressing agent is administered prior to the administration of an anticancer agent. In some embodiments, the suppressing agent is administered prior to the administration of an anticancer agent and prior to the onset of an adverse event. In some embodiments, the suppressing agent is administered within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of an anticancer therapy.

In some embodiments, the suppressing agent is administered at the same time (or substantially at the same time) as an anticancer agent. In some embodiments, the suppressing agent and the anticancer agent are administered at the same time as a single composition. In some embodiments, the suppressing agent and the anticancer agent are administered at the same time as more than one composition.

As used herein, the term “round” refers to a course of treatment. In general, each round of a treatment is alternated with a period of time during which the subject does not receive the treatment. In some embodiments, the subject may receive more than one round of an anticancer therapy. In some embodiments, the subject may receive more than one round of an anticancer therapy. It should be appreciated that a round of treatment (e.g., of an anticancer therapy, suppressing agent) may involve one or more doses of the treatment. In some embodiments, each round of the treatment may use the same therapeutic (e.g., the same anticancer agent or suppressing agent), or different therapeutics (e.g., different anticancer agents or suppressing agents).

In some embodiments, a round of anticancer therapy may last 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or longer.

In some embodiments, the suppressing agent is administered after one round of anticancer therapy. In some embodiments, the suppressing agent is administered immediately after completion of the round of the anticancer therapy. In some embodiments, the suppressing agent is administered within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more after completion of the round of anticancer therapy.

In some embodiments, the suppressing agent is administered prior to completing one round of anticancer therapy. In some embodiments, the suppressing agent is administered at the concurrently with the round of anticancer therapy. In some embodiments, the suppressing agent is administered within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more after the initiation of the round of anticancer therapy.

Also within the scope of the present disclosure are multiple or repeated administrations (e.g., doses) of any of the compositions described herein (e.g., anticancer therapy, suppressing agents, and/or combination therapies). In some embodiments, administration of the anticancer therapy is repeated two or more (e.g., 3, 4, 5, 6, or more) times. In some embodiments, administration of the suppressing agent is repeated two or more (e.g., 3, 4, 5, 6, or more) times. In some embodiments, administration of both the anticancer therapy and the suppressing agent are repeated two or more (e.g., 3, 4, 5, 6, or more) times. In some embodiments, the anticancer therapy and/or suppressing agents are administered to the subject at a regular interval, e.g., every six months.

The compositions and methods described herein may be utilized in conjunction with other types of therapy (i.e., combination treatment), such as additional therapeutic agents. Examples of additional combination therapies include, without limitation, surgery, radiation, gene therapy, and administration of additional therapeutic agents, such as chemotherapeutics, antibiotics, antivirals, anti-fungals, anti-parasitics, immunomodulatory agents, anti-inflammatory agents. In general, combination therapies can be administered simultaneously or sequentially (in any order) with the compositions and methods described herein. In some embodiments, any of the compositions described herein is administered simultaneously with one or more additional therapeutic agents, for example in a single dose or in multiple doses that are administered at substantially the same time.

In some embodiments, the compositions described herein are administered to a subject concomitantly with one or more additional therapeutic agents. In some embodiments, the compositions described herein are administered to a subject followed by administration of one or more additional therapeutic agent. In some embodiments, any of the compositions described herein is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the one or more additional therapeutic agent. Alternatively, in some embodiments, one or more therapeutic agent administered to a subject followed by administration of any of the compositions described herein. In some embodiments, one or more therapeutic agent is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of any the compositions described herein.

In some embodiments, the subject has not received a dose of an antibiotic prior to administration of the suppressing agent (e.g. suppressing live bacterial product). In some embodiments, the subject has not been administered an antibiotic at least 1, at least 2, at least 3, at least 5, at least 10, at least 15, at least 20, at least 25, at least 30, at least 60, at least 90, at least 120, at least 180, at least 360 days or more prior to administration of the compositions provided herein.

In some embodiments, the subject may be administered one or more doses of an antibiotic prior to or concurrently with a suppressing agent (e.g., suppressing live bacterial product). Antibiotics may be administered for a variety of reasons. For instance, antibiotics may be administered to remove bacterial species from the colon and/or intestine prior to administration of the suppressing agents provided herein. In some embodiments, antibiotics are administered to increase the ability of the bacterial strains of the suppressing live bacterial products to engraft in the colon and/or intestine. Antibiotics may also be administered to suppress unwanted infections in the case of anticancer therapy. In some instances, antibiotics may be administered as a treatment method for an infectious disease.

In some embodiments, the subject is administered a single dose of an antibiotic prior to the suppressing agent. In some embodiments, the subject is administered multiple doses of an antibiotic prior to the suppressing agent. In some embodiments, the subject is administered at least 2, 3, 4, 5 or more doses of an antibiotic prior to the suppressing agent. In some embodiments, the subject is administered a dose of an antibiotic at substantially the same time as the suppressing agent. Examples of antibiotics that can be administered include, without limitation, kanamycin, gentamicin, colistin, metronidazole, vancomycin, clindamycin, fidaxomicin, and cefoperazone.

Any of the methods described herein may also involve determining whether a specific bacteria or bacterial species is present in the subject, for example, prior to administering a suppressing agent. In some embodiments, the method involves determining whether a specific bacteria or bacterial species from an anticancer live bacterial product is present in the subject (e.g., in the intestine of the subject) prior to administering a suppressing agent. In some embodiments, the method involves determining whether a specific pathogenic bacteria or bacterial species is present in the subject (e.g., in the intestine of the subject) prior to administering a suppressing agent. In some embodiments, if the specific bacteria or bacterial species is detecting in the subject or a sample obtained from the subject, the subject is administered one or more doses of an antibiotic prior to the suppressing agent.

In general, the presence of a specific bacteria or bacterial species or the composition of the bacterial population of the subject may be determined by assessing a sample obtained from the subject, such as a fecal sample.

In some embodiments, the subject is treated with one or more antibiotics (e.g., one or more doses) prior to administration of the suppressing agent. In some embodiments, the antibiotics is administered immediately prior to the administration of the suppressing agent. In some embodiments, an antibiotic is administered within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks or more prior to the administration of the suppressing agent.

### Compositions

Also within the scope of the present disclosure are compositions, e.g., compositions for administering to a subject, such as pharmaceutical compositions. In some embodiments, the composition comprises any one or more anticancer agent, as described herein (e.g., an anticancer live bacterial product). In some embodiments, the composition comprises any one or more suppressing agent (e.g., a suppressing live bacterial product), as described herein. In some embodiments, the composition comprises any one or more anticancer agent, as described herein (e.g., an anticancer live bacterial product), and any one or more suppressing agent (e.g., a suppressing live bacterial product), as described herein.

In one aspect, the disclosure provides pharmaceutical compositions comprising any one of anticancer agents and/or any one of the suppressing agents described herein. In some embodiments of the compositions provided herein, the composition is a pharmaceutical composition. In some embodiments, the pharmaceutical composition comprises a pharmaceutical acceptable excipient. In some embodiments, the pharmaceutical composition is formulated for oral administration. In some embodiments, the pharmaceutical composition is formulated for rectal administration. In some embodiments, the pharmaceutical composition is formulated for delivery to the intestine. In some embodiments, the pharmaceutical composition is formulated for delivery to the colon.

In some embodiments, the composition or pharmaceutical composition include a live bacterial product, such as an anticancer live bacterial product and/or a suppressing live bacterial product. In some embodiments, the live bacterial product may be lyophilized. In some embodiments, the pharmaceutical composition is in the form of a capsule. In some embodiments, the pharmaceutical composition further comprises a pH sensitive composition comprising one or more enteric polymers.

Any of the compositions described herein, including the pharmaceutical compositions and food products comprising the live bacterial products, the live bacterial product may contain bacterial strains in any form, for example in an aqueous form, such as a solution or a suspension, embedded in a semi-solid form, in a powdered form or freeze-dried form. In some embodiments, the composition or the bacterial strains of the live bacterial product are lyophilized. In some embodiments, a subset of the bacterial strains of the live bacterial product is lyophilized. Methods of lyophilizing compositions, specifically compositions comprising bacteria, are well known in the art; See, e.g., U.S. Pat. Nos. 3,261,761; 4,205,132; PCT Publications WO 2014/029578 and WO 2012/098358, which are all herein incorporated by reference in their entirety. The bacteria may be lyophilized as a combination and/or the bacteria may be lyophilized separately and combined prior to administration. A bacterial strain may be combined with a pharmaceutical excipient prior to combining it with the other bacterial strain or multiple lyophilized bacteria may be combined while in lyophilized form and the mixture of bacteria, once combined may be subsequently be combined with a pharmaceutical excipient. In some embodiments, the bacterial strain is a lyophilized cake. In some embodiments, the compositions comprising the one or more bacterial strains are a lyophilized cake.

In some embodiments, one or more of the bacterial strains of the compositions, including pharmaceutical compositions and food products, has been spray-dried. In some embodiments, a subset of the bacterial strains is spray-dried. The process of spray-drying refers to production of dry powder from a liquid comprising bacterial compositions. (See, e.g., Ledet et al., Spray-Drying of Pharmaceuticals in “Lyophilized Biologics and Vaccines” pages 273-294, Springer). In general, the process involves rapidly drying the bacterial compositions with a hot gas. A bacterial strain may be combined with a pharmaceutical excipient prior to combining it with the other bacterial strains or multiple spray-dried bacterial strains may be combined while in spray-dried form and the mixture of bacterial strains, once combined may be subsequently be combined with a pharmaceutical excipient.

The bacterial strains of the live bacterial products can be manufactured using fermentation techniques well known in the art. In some embodiments, the active ingredients are manufactured using anaerobic fermenters, which can support the rapid growth of anaerobic bacterial species. The anaerobic fermenters may be, for example, stirred tank reactors or disposable wave bioreactors. Culture media such as BL media and EG media, or similar versions of these media devoid of animal components, can be used to support the growth of the bacterial species. The bacterial product can be purified and concentrated from the fermentation broth by traditional techniques, such as centrifugation and filtration, and can optionally be dried and lyophilized by techniques well known in the art.

In some embodiments, the live bacterial product may be formulated for administration as a pharmaceutical composition. The term “pharmaceutical composition” as used herein means a product that results from the mixing or combining of at least one active ingredient, such as any of the suppressing agents described herein and/or any of the anticancer agents described herein, and one or more inactive ingredients, which may include one or more pharmaceutically acceptable excipient.

An “acceptable” excipient refers to an excipient that must be compatible with the active ingredient and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Pharmaceutical compositions of the invention can be prepared in accordance with methods well known and routinely practiced in the art (see e.g., Remington: The Science and Practice of Pharmacy, Mack Publishing Co. 20th ed. 2000). The pharmaceutical compositions described herein may further comprise any carriers or stabilizers in the form of a lyophilized formulation or an aqueous solution. Acceptable excipients, carriers, or stabilizers may include, for example, buffers, antioxidants, preservatives, polymers, chelating reagents, and/or surfactants. Pharmaceutical compositions are preferably manufactured under GMP conditions. The pharmaceutical compositions can be used orally, nasally or parenterally, for instance, in the form of capsules, tablets, pills, sachets, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like.

In some embodiments, the anticancer agents and/or suppressing agents are formulated for delivery to the intestines (e.g., the small intestine and/or the colon). In some embodiments, the anticancer agent and/or suppressing agent is a live bacterial product, may be formulated with an enteric coating that increases the survival of the bacteria through the harsh environment in the stomach. The enteric coating is one which resists the action of gastric juices in the stomach so that the bacteria of the live bacterial product therein will pass through the stomach and into the intestines. The enteric coating may readily dissolve when in contact with intestinal fluids, so that the bacteria enclosed in the coating will be released in the intestinal tract. Enteric coatings may consist of polymer and copolymers well known in the art, such as commercially available EUDRAGIT (Evonik Industries). (See e.g., Zhang, AAPS PharmSciTech (2016) 17 (1), 56-67).

The one or more anticancer agents (e.g., an anticancer live bacterial product) and one or more suppressing agents (e.g., a suppressing live bacterial product) may also be formulated for rectal delivery to the intestine (e.g., the colon). Thus, in some embodiments, the anticancer agents and/or suppressing agents may be formulated for delivery by suppository, colonoscopy, endoscopy, sigmoidoscopy, or enema. A pharmaceutical preparation or formulation and particularly a pharmaceutical preparation for oral administration, may include an additional component that enables efficient delivery of the compositions of the disclosure to the intestine (e.g., the colon). A variety of pharmaceutical preparations that allow for the delivery of the compositions to the intestine (e.g., the colon) can be used. Examples thereof include pH sensitive compositions, more specifically, buffered sachet formulations or enteric polymers that release their contents when the pH becomes alkaline after the enteric polymers pass through the stomach. When a pH sensitive composition is used for formulating the pharmaceutical preparation, the pH sensitive composition is preferably a polymer whose pH threshold of the decomposition of the composition is between about 6.8 and about 7.5. Such a numeric value range is a range in which the pH shifts toward the alkaline side at a distal portion of the stomach, and hence is a suitable range for use in the delivery to the colon. It should further be appreciated that each part of the intestine (e.g., the duodenum, jejunum, ileum, cecum, colon, and rectum), has different biochemical and chemical environment. For instance, parts of the intestines have different pHs, allowing for targeted delivery by compositions that have a specific pH sensitivity. Thus, the compositions provided herein may be formulated for delivery to the intestine or specific parts of the intestine (e.g., the duodenum, jejunum, ileum, cecum, colon, and rectum) by providing formulations with the appropriate pH sensitivity. (See e.g., Villena et al., Int J Pharm (2015) 487 (1-2): 314-9).

Another embodiment of a pharmaceutical preparation useful for delivery of the compositions to the intestine (e.g., the colon) is one that ensures the delivery to the colon by delaying the release of the contents (e.g., the anticancer agent and/or suppressing agent) by approximately 3 to 5 hours, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Additional examples of pharmaceutical compositions that allow for the delivery to the intestine (e.g., the colon) include bioadhesive compositions which specifically adhere to the colonic mucosal membrane (for example, a polymer described in the specification of U.S. Pat. No. 6,368,586) and compositions into which a protease inhibitor is incorporated for protecting particularly a biopharmaceutical preparation in the gastrointestinal tracts from decomposition due to an activity of a protease.

Another example of a system enabling the delivery to the intestine (e.g., the colon) is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

A further example of a system enabling the delivery of a composition to the intestine (e.g., the colon), is a composition that includes a coating that can be removed by an enzyme present in the gut (e.g., the colon), such as, for example, a carbohydrate hydrolase or a carbohydrate reductase. Such a system is not particularly limited, and more specific examples thereof include systems which use food components such as non-starch polysaccharides, amylose, xanthan gum, and azopolymers.

The compositions provided herein can also be delivered to specific target areas, such as the intestine, by delivery through an orifice (e.g., a nasal tube) or through surgery. In addition, the compositions provided herein that are formulated for delivery to a specific area (e.g., the cecum or the colon), may be administered by a tube (e.g., directly into the small intestine). Combining mechanical delivery methods such as tubes with chemical delivery methods such as pH specific coatings, allow for the delivery of the compositions provided herein to a desired target area (e.g., the cecum or the colon).

The compositions comprising anticancer agents and/or suppressing agents are formulated into pharmaceutically acceptable dosage forms by conventional methods known to those of skill in the art. Dosage regimens are adjusted to provide the optimum desired response (e.g., the prophylactic or therapeutic effect). In some embodiments, the dosage form of the composition is a tablet, pill, capsule, powder, granules, solution, or suppository. In some embodiments, the pharmaceutical composition is formulated for oral administration. In some embodiments, the pharmaceutical composition comprises a live bacterial product (e.g., an anticancer live bacterial product and/or a suppressing live bacterial product) and is formulated such that the bacteria of the live bacterial product, or a portion thereof, remain viable after passage through the stomach of the subject. In some embodiments, the pharmaceutical composition is formulated for rectal administration, e.g. as a suppository. In some embodiments, the pharmaceutical composition is formulated for delivery to the intestine or a specific area of the intestine (e.g., the colon) by providing an appropriate coating (e.g., a pH specific coating, a coating that can be degraded by target area specific enzymes, or a coating that can bind to receptors that are present in a target area).

Dosages of the active ingredients in the pharmaceutical compositions of the present invention can be varied so as to obtain an amount of the active ingredient which is effective to achieve the desired pharmaceutical response for a particular subject, composition, and mode of administration, without being toxic or having an adverse effect on the subject. The selected dosage level depends upon a variety of factors including the activity of the particular compositions of the present invention employed, the route of administration, the time of administration, the duration of the treatment, other drugs, compounds and/or materials used in combination with the particular compositions employed, the age, sex, weight, condition, general health and prior medical history of the subject being treated, and like factors.

A physician, veterinarian or other trained practitioner, can start doses of the pharmaceutical composition at levels lower than that required to achieve the desired therapeutic effect and gradually increase the dosage until the desired effect (e.g., treatment of the cancer and/or reduction or elimination of adverse events caused by the anticancer therapy) is achieved. In general, effective doses of the compositions of the present invention, for the prophylactic treatment of groups of people as described herein vary depending upon many different factors, including routes of administration, physiological state of the subject, whether the subject is human or an animal, other medications administered, and the therapeutic effect desired. Dosages need to be titrated to optimize safety and efficacy. In some embodiments, the dosing regimen entails oral administration of a dose of any of the compositions described herein. In some embodiments, the dosing regimen entails oral administration of multiple doses of any of the compositions described herein. In some embodiments, the any of the compositions described herein are administered the subject once, twice, 3 times, 4 times, 5 times, 6 times, 7 times, 8 times, 9 times, or at least 10 times.

The compositions, including the pharmaceutical compositions disclosed herein, include compositions that may comprise an anticancer agent and/or a suppressing agent that may be a live bacterial product. The quantity of bacteria in the live bacterial product or in the composition may be expressed in weight, number of bacteria and/or CFUs (colony forming units). In some embodiments, the live bacterial product or pharmaceutical composition comprising a live bacterial product contain about 10, about 10², about 10³, about 10⁴, about 10⁵, about 10⁶, about 10⁷, about 10⁸, about 10⁹, about 10¹⁰, about 10¹¹, about 10¹², about 10¹³ or more of each of the bacteria of the live bacterial product per dosage amount. In some embodiments, the live bacterial product or pharmaceutical composition comprising a live bacterial product contain about 10, about 10², about 10³, about 10⁴, about 10⁵, about 10⁶, about 10⁷, about 10⁸, about 10⁹, about 10¹⁰, about 10¹¹, about 10¹², about 10¹³ or more total bacteria per dosage amount. It should further be appreciated that the bacteria of the live bacterial products may be present in different amounts. Thus, for instance, as a non-limiting example, a live bacterial product may include 10³ of bacteria A, 10⁴ of bacteria B and 10⁶ of bacteria C. In some embodiments, the live bacterial product or pharmaceutical composition comprising a live bacterial product contain about 10, about 10², about 10³, about 10⁴, about 10⁵, about 10⁶, about 10⁷, about 10⁸, about 10⁹, about 10¹⁰, about 10¹¹, about 10¹², about 10¹³ or more CFUs of each of the bacteria in the live bacterial product per dosage amount. In some embodiments, the live bacterial product or pharmaceutical composition comprising a live bacterial product contain about 10¹, about 10², about 10³, about 10⁴, about 10⁵, about 10⁶, about 10⁷, about 10⁸, about 10⁹, about 10¹⁰, about 10¹¹, about 10¹², about 10¹³ or more CFUs in total for all of the bacteria combined per dosage amount. As discussed above, bacteria of the live bacterial products may be present in different amounts. In some embodiments, the live bacterial product or pharmaceutical composition comprising a live bacterial product contain about 10⁻⁷, about 10⁻⁶, about 10⁻⁵, about 10⁴, about 10⁻³, about 10⁻², about 10⁻¹ or more grams of each of the bacteria in the composition per dosage amount. In some embodiments, the live bacterial product or pharmaceutical composition comprising a live bacterial product contain about 10⁻⁷, about 10⁻⁶, about 10⁻⁵, about 10⁴, about 10⁻³, about 10⁻², about 10⁻¹ or more grams in total for all of the bacteria combined per dosage amount. In some embodiment, the dosage amount is one administration device (e.g., one table, pill or capsule). In some embodiment, the dosage amount is the amount that is administered in a particular period (e.g., one day or one week).

In some embodiments, the live bacterial product or pharmaceutical composition comprising a live bacterial product described herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² of each of the bacteria of the live bacterial product per dosage amount. In some embodiments, the live bacterial product or pharmaceutical composition comprising a live bacterial product contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria per dosage amount.

In some embodiments, the live bacterial product or pharmaceutical composition comprising a live bacterial product contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² CFUs of each of the bacteria of the live bacterial product per dosage amount. In some embodiments, the live bacterial product or pharmaceutical composition comprising a live bacterial product contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total CFUs per dosage amount.

In some embodiments, the live bacterial product or pharmaceutical composition comprising a live bacterial product contain between 10⁻⁷ and 10⁻¹, between 10⁻⁶ and 10⁻¹, between 10⁻⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁻⁵ and 10⁻², between 10⁴ and 10⁻², between 10⁻³ and 10⁻², between 10⁻⁷ and 10⁻³, between 10⁻⁶ and 10⁻³, between 10⁻⁵ and 10⁻³, between 10⁻⁴ and 10⁻³, between 10⁻⁷ and 10⁴, between 10⁻⁶ and 10⁴, between 10⁻⁵ and 10⁴, between 10⁻⁷ and 10⁻⁵, between 10⁻⁶ and 10⁻⁵, or between 10⁻⁷ and 10⁻⁶ grams of each of the bacteria in the composition per dosage amount. In some embodiments, the pharmaceutical compositions disclosed herein contain between 10⁻⁷ and 10⁻¹, between 10⁻⁶ and 10⁻¹, between 10⁻⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁻⁵ and 10⁻², between 10⁻⁴ and 10⁻², between 10⁻³ and 10⁻², between 10⁻⁷ and 10⁻³, between 10⁻⁶ and 10⁻³, between 10⁻⁵ and 10⁻³, between 10⁻⁴ and 10⁻³, between 10⁻⁷ and 10⁻⁴, between 10⁻⁶ and 10⁻⁴, between 10⁻⁵ and 10⁻⁴, between 10⁻⁷ and 10⁻⁵, between 10⁻⁶ and 10⁻⁵, or between 10⁻⁷ and 10⁻⁶ grams of all of the bacteria combined per dosage amount.

In one aspect, the disclosure provides a food product comprising any of the compositions provided herein and a nutrient. Also with the scope of the present disclosure are food products comprising any of the live bacterial products described herein and a nutrient. Food products are, in general, intended for the consumption of a human or an animal. Any of the live bacterial products described herein may be formulated as a food product. In some embodiments, the live bacterial products are formulated as a food product in spore form. In some embodiments, the live bacterial products are formulated as a food product in vegetative form. In some embodiments, the food product comprises both vegetative bacteria and bacteria in spore form. The compositions disclosed herein can be used in a food or beverage, such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Non-limiting examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products such as Western confectionery products including biscuits, cookies, and the like, Japanese confectionery products including steamed bean-jam buns, soft adzuki-bean jellies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

Food products containing live bacterial products described herein may be produced using methods known in the art and may contain the same amount of bacteria (e.g., by weight, amount or CFU) as the pharmaceutical compositions provided herein. Selection of an appropriate amount of bacteria in the food product may depend on various factors, including for example, the serving size of the food product, the frequency of consumption of the food product, the specific bacterial strains contained in the food product, the amount of water in the food product, and/or additional conditions for survival of the bacteria in the food product.

Examples of food products which may be formulated to contain any of the live bacterial products described herein include, without limitation, a beverage, a drink, a bar, a snack, a dairy product, a confectionery product, a cereal product, a ready-to-eat product, a nutritional formula, such as a nutritional supplementary formulation, a food or beverage additive.

### Kits

Also within the scope of the present disclosure are kits for use in the methods described herein. Such kits may include one or more containers comprising an agent for detecting a biomarker of an adverse event caused by an anticancer therapy and any of the suppressing agents described herein. In some embodiments, the kit may also include one or more containers comprising any of the anticancer agents described herein. In some embodiments, the kit may also include one or more antibiotics.

In some embodiments, the kit contains one or more agents for detecting and/or measuring the amount of a biomarker in a sample from a subject (e.g., a cancer patient). In some embodiments, the agent that detects or measures the biomarker can comprise one or more binding agents that specifically bind to the biomarker. In some embodiments, the binding agent is an antibody that specifically binds to the biomarker. In some embodiments, the binding agent is part of a reporter system, such as a receptor on a cell that binds to the biomarker and induces expression of a gene encoding a reporter molecule. In some embodiments, the kit also contains a standard or control sample to which the amount of the biomarker in the sample(s) obtained from the subject may be compared.

In some embodiments, the kit may be for carrying out any of the companion diagnostic or prognostic methods described herein. In some embodiments, the kit may be for identifying a subject as a subject in need of a suppressing agent as well as for administering the suppressing agent. In some embodiments, the kit may be for anticancer therapy and include one or more anticancer agent as well as an agent for detecting a biomarker of an adverse event caused by an anticancer therapy and any of the suppressing agents described herein for administering to the subject. In some embodiments, the kit also includes an antibiotic, for example, an antibiotic for administering to the subject prior to administration of the suppressing agent.

In some embodiments, the biomarker is analyzed by detecting the presence of a nucleic acid encoding the biomarker, by measuring the level (amount) of a nucleic acid encoding the marker, and/or a specific cell type in which the nucleic acid encoding the marker is expressed. In some embodiments, the kit includes one or more reagents for the isolation of nucleic acids (e.g., RNA) from a sample obtained from subject.

In some embodiments, the kits further comprise a detection agent (e.g., an antibody binding to the binding agent) for detecting binding of the agent to the biomarker in the sample. The detection agent can be conjugated to a label. In some embodiments, the detection agent is an antibody that specifically binds to at least one of the binding agents. In some embodiments, the binding agent comprises a tag that can be identified and, directly or indirectly, bound by a detection agent.

In some embodiments, the kit includes one or more suppressing agents for administering to the subject. For example, in some embodiments, the kit may include any of the suppressing live bacterial products described herein or other suppressing agent (e.g., a steroid or infliximab).

In some embodiments, the kit includes one or more anticancer agents for administering to the subject. For example, in some embodiments, the kit may include one or more immune checkpoint inhibitor (e.g., PD-1 inhibitor, PD-L1 inhibitor, CTLA-4 inhibitor). In some embodiments, the kit includes one or more doses of the anticancer agent.

In some embodiments, the kit or device further includes a support member. In some embodiments, the support member is a membrane, such as a nitrocellulose membrane, a polyvinylidene fluoride (PVDF) membrane, or a cellulose acetate membrane. In some examples, the immunoassay may be in a Western blot assay format or a lateral flow assay format.

In some embodiments, the support member is a multi-well plate, such as an ELISA plate. In some embodiments, the immunoassays described herein can be carried out on high throughput platforms. In some embodiments, multi-well plates, e.g., 24-, 48-, 96-, 384- or greater well plates, may be used for high throughput detection assays.

In the kit or detecting device, one or more of the agents for detecting a biomarker may be immobilized on a support member, which can be a membrane, a bead, a slide, or a multi-well plate. Selection of an appropriate support member for the immunoassay will depend on various factor such as the number of samples and method of detecting the signal released from label conjugated to the second agent.

The kit can also comprise one or more buffers as described herein but not limited to a coating buffer, a blocking buffer, a wash buffer, and/or a stopping buffer.

In some embodiments, the kit can comprise instructions for use in accordance with any of the methods described herein. The instructions relating to the use of the kit generally include information as to the amount of each component and suitable conditions for performing an assay for the detection of a biomarker and/or instructions relating to the use of the kit generally include information as to the amount of each component and suitable conditions for administering any of the components to the subject. The components in the kits may be in unit doses, bulk packages (e.g., multi-dose packages), or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

The label or package insert indicates that the kit is used for detecting the level of one or more biomarkers associated with an adverse event, selecting a treatment, and/or diagnostic purposes. Instructions may be provided for practicing any of the methods described herein.

The kits of this present disclosure are in suitable packaging. Suitable packaging includes, but is not limited to, vials, bottles, jars, flexible packaging (e.g., sealed Mylar or plastic bags), and the like.

Kits may optionally provide additional components such as interpretive information, such as a control and/or standard or reference sample. Normally, the kit comprises a container and a label or package insert(s) on or associated with the container. In some embodiments, the present disclosure provides articles of manufacture comprising contents of the kits described above.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Unless otherwise defined herein, scientific and technical terms used in connection with the present disclosure shall have the meanings that are commonly understood by those of ordinary skill in the art. Further, unless otherwise required by context, singular terms shall include pluralities and plural terms hall include the singular. The methods and techniques of the present disclosure are generally performed according to conventional methods well-known in the art. Generally, nomenclatures used in connection with, and techniques of biochemistry, enzymology, molecular and cellular biology, microbiology, virology, cell or tissue culture, genetics and protein and nucleic chemistry described herein are those well-known and commonly used in the art. The methods and techniques of the present disclosure are generally performed according to conventional methods well known in the art and as described in various general and more specific references that are cited and discussed throughout the present specification unless otherwise indicated.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Example 1: Anticancer Treatment and the Suppression of Side Effects

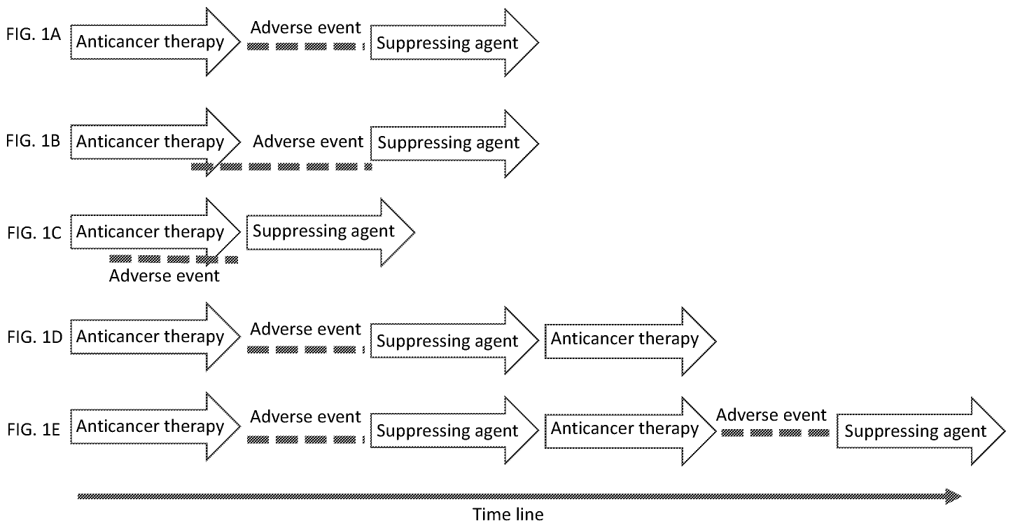

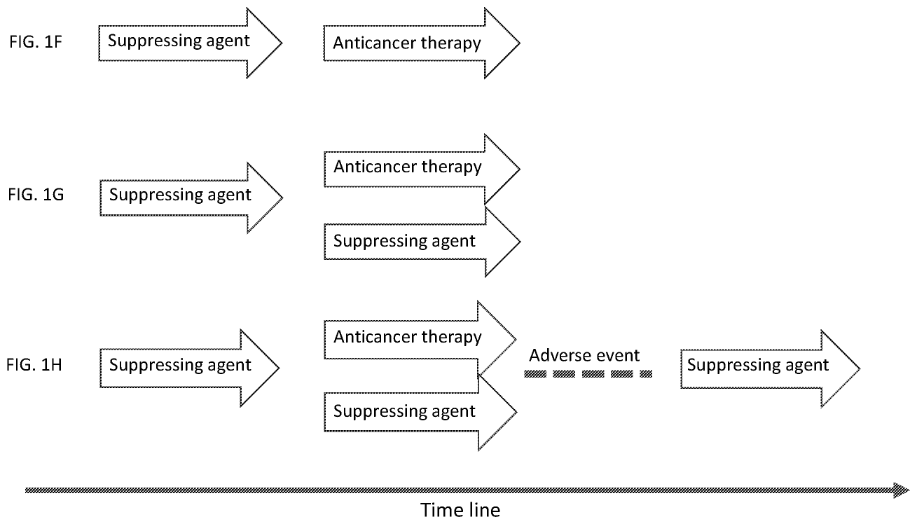

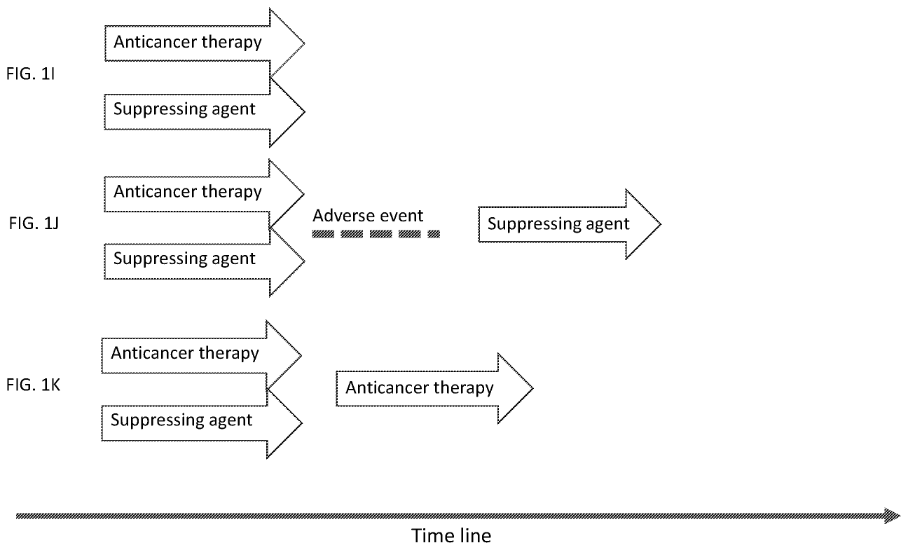

As shown in FIGS. 1A-1K, the methods and compositions described herein may be used in any of a number of non-limiting example cancer treatment regimens.

FIG. 1A shows a treatment course in which the subject receives an anticancer therapy, then experiences an adverse event and is subsequently administered a suppressing agent.

FIG. 1B shows a treatment course in which the subject receives an anticancer therapy and experiences an adverse event, which begins during the course of the anticancer therapy. The subject is subsequently administered a suppressing agent.

FIG. 1C shows a treatment course in which the subject receives an anticancer therapy and experiences an adverse event shortly after beginning the anticancer therapy. The subject is subsequently administered a suppressing agent.

FIG. 1D shows a treatment course in which the subject receives an anticancer therapy, then experiences an adverse event and is administered a suppressing agent. The subject is then able to undergo a subsequent round of anticancer therapy.

FIG. 1E shows a treatment course in which the subject receives an anticancer therapy, then experiences an adverse event and is subsequently administered a suppressing agent. The subject then undergoes a subsequent round of anticancer therapy and experiences a further adverse event, following by an additional administration of a suppressing agent.

FIG. 1F shows a treatment course in which the subject receives a suppressing agent, followed by anticancer therapy.

FIG. 1G shows a treatment course in which the subject receives a suppressing agent, followed by administration of a second round of suppressing agent at the same as receiving anticancer therapy.

FIG. 1H shows a treatment course in which the subject receives a suppressing agent, followed by administration of a second round of suppressing agent at the same as receiving anticancer therapy. The subject subsequently experiences an adverse event and receives an additional administration of a suppressing agent.

FIG. 1I shows a treatment course in which the subject receives a suppressing agent at the same as receiving anticancer therapy.

FIG. 1J shows a treatment course in which the subject receives a suppressing agent at the same as receiving anticancer therapy. The subject subsequently experiences an adverse event and receives an additional administration of a suppressing agent.

FIG. 1K shows a treatment course in which the subject receives a suppressing agent, at the same as receiving anticancer therapy. The subject subsequently undergoes a subsequent round of anticancer therapy.

What is claimed is:

1. A method of treatment comprising administering to a subject undergoing anticancer therapy a suppressing agent that suppresses an adverse event caused by the anticancer therapy.

2. A method of treatment comprising administering a pharmaceutically effective amount of anticancer therapy to a subject in need thereof and administering to the subject a suppressing agent that suppresses an adverse event caused by the anticancer therapy.

3. A method of treatment comprising administering to a subject in need thereof a suppressing agent followed by a pharmaceutically effective amount of anticancer therapy.

4. A method of treatment comprising administering to a subject in need thereof a combination of a suppressing agent and a pharmaceutically effective amount of anticancer therapy.

5. A method of treatment comprising administering a pharmaceutically effective amount of anticancer therapy to a subject in need thereof, determining if an adverse event occurs in the subject, and administering to the subject a suppressing agent that suppresses the adverse event.

6. A method of treatment comprising administering a pharmaceutically effective amount of anticancer therapy to a subject in need thereof, determining if an adverse event occurs in the subject, wherein if an adverse event occurs, administering to the subject a suppressing agent that suppresses the adverse event.

7. The method of any one of the preceding claims, wherein the anticancer therapy comprises the administration of one or more anticancer agents.

8. The method of claim 7, wherein the anticancer agent is a chemotherapy agent.

9. The method of claim 7, wherein the anticancer agent is a cancer immunotherapy agent.

10. The method of claim 9, wherein the cancer immunotherapy agent is an immune checkpoint inhibitor.

11. The method of claim 10, wherein the immune checkpoint inhibitor is a PD-1 inhibitor, PD-L1 inhibitor, or CTLA-4 inhibitor.

12. The method of claim 11, wherein the immune checkpoint inhibitor is a PD-1 inhibitor.

13. The method of claim 12, wherein the PD-1 inhibitor is nivolumab or pembrolizumab.

14. The method of claim 11, wherein the immune checkpoint inhibitor is a PD-L1 inhibitor.

15. The method of claim 14, wherein the PD-L1 inhibitor is atezolizumab, avelumab or durvalumab.

16. The method of claim 11, wherein the immune checkpoint inhibitor is a CTLA-4 inhibitor.

17. The method of claim 16, wherein the CTLA-4 inhibitor is ipilimumab or tremelimumab.

18. The method of any one of the preceding claims, wherein the anticancer therapy comprises the administration of one or more cytokines.

19. The method of claim 18, wherein the cytokine is interferon-alpha, tumor necrosis factor, IL-2, IL-12, IL-15, or IL-21.

20. The method of any one of the preceding claims, wherein the anticancer therapy comprises the administration of one or more costimulatory agents.

21. The method of claim 20, wherein the costimulatory agent is a CD-28, OX-40, 4-1BB, or CD40 antibody.

22. The method of any one of the preceding claims, wherein the anticancer therapy comprises the administration of an anticancer live bacterial product.

23. The method of any one of the preceding claims, wherein the anticancer therapy comprises the administration of an immune checkpoint inhibitor and an anticancer live bacterial product.

24. The method of claim 22 or 23, wherein the anticancer live bacterial product increases the efficacy of the immune checkpoint inhibitor.

25. The method of any one of claims 22-24, wherein the anticancer live bacterial product induces CD8+ T-cells.

26. The method of any one of claims 22-25, wherein the anticancer live bacterial product induces Th17 cells.

27. The method of any one of claims 22-26, wherein the anticancer live bacterial product induces Th1 cells.

28. The method of any one of claims 22-27, wherein the anticancer live bacterial product comprises bacterial strains of species associated with increased efficacy in anticancer treatment.

29. The method of any one of the preceding claims, wherein the anticancer therapy comprises the administration of a PD-1 inhibitor and an anticancer live bacterial product that induces CD8+ T-cells.

30. The method of any one of the preceding claims, wherein the anticancer therapy comprises the administration of a PD-1 inhibitor and an anticancer live bacterial product that induces Th17 cells.

31. The method of any one of the preceding claims, wherein the anticancer therapy comprises the administration of a PD-1 inhibitor and an anticancer live bacterial product that induces Th1 cells.

32. The method of any one of the preceding claims, wherein the anticancer therapy comprises the administration of a PD-1 inhibitor and an anticancer live bacterial product that comprises bacterial strains of species associated with increased efficacy in anticancer treatment with a PD-1 inhibitor.

33. The method of any one of the preceding claims, wherein the anticancer therapy comprises the administration of a PD-L1 inhibitor and an anticancer live bacterial product that induces CD8+ T-cells.

34. The method of any one of the preceding claims, wherein the anticancer therapy comprises the administration of a PD-L1 inhibitor and an anticancer live bacterial product that induces Th17 cells.

35. The method of any one of the preceding claims, wherein the anticancer therapy comprises the administration of a PD-L1 inhibitor and an anticancer live bacterial product that induces Th1 cells.

36. The method of any one of the preceding claims, wherein the anticancer therapy comprises the administration of a PD-L1 inhibitor and an anticancer live bacterial product that comprises bacterial strains of species associated with increased efficacy in anticancer treatment with a PD-L1 inhibitor.

37. The method of any one of the preceding claims, wherein the anticancer therapy comprises the administration of a CTLA-4 inhibitor and an anticancer live bacterial product that induces CD8+ T-cells.

38. The method of any one of the preceding claims, wherein the anticancer therapy comprises the administration of a CTLA-4 inhibitor and an anticancer live bacterial product that induces Th17 cells.

39. The method of any one of the preceding claims, wherein the anticancer therapy comprises the administration of a CTLA-4 inhibitor and an anticancer live bacterial product that induces Th1 cells.

40. The method of any one of the preceding claims, wherein the anticancer therapy comprises the administration of a CTLA-4 inhibitor and an anticancer live bacterial product that comprises bacterial strains of species associated with increased efficacy in anticancer treatment with a CTLA-4 inhibitor.

41. The method of any one of the preceding claims, wherein the suppressing agent is an agent that suppresses the immune response.

42. The method of any one of the preceding claims, wherein the suppressing agent is a suppressing live bacterial product.

43. The method of claim 42, wherein the suppressing live bacterial product induces regulatory T cells.

44. The method of claims 42 and 43, wherein the suppressing live bacterial product comprises bacterial strains belonging to Clostridium cluster XIVa and/or Clostridium cluster IV.

45. The method of any one claims 42-44, wherein the suppressing live bacterial product comprises bacterial strains belonging to Clostridium cluster XIVa.

46. The method of any one claims 42-45, wherein the suppressing live bacterial product is VE-202.

47. The method of any one of the preceding claims, wherein the adverse event is an undesired immune response.

48. The method of any one of the preceding claims, wherein the adverse event is colitis.

49. The method of any one of the preceding claims, wherein the adverse event is dermatological toxicity.

50. The method of any one of the preceding claims, wherein the adverse event is diarrhea.

51. The method of any one of the preceding claims, wherein the adverse event is hepatotoxicity.

52. The method of any one of the preceding claims, wherein the adverse event is hypophysitis.

53. The method of any one of the preceding claims, wherein the adverse event is autoimmune thyroid disease.

54. The method of any one of the preceding claims, wherein the suppressing agent is administered after the occurrence of the adverse event.

55. The method of any one of the preceding claims, wherein the suppressing agent is administered prior to the occurrence of the adverse event.

56. The method of any one of the preceding claims, wherein the method further comprises repeating the anticancer therapy.

57. The method of any one of the preceding claims, wherein the method further comprises repeating the administration of the suppressing agent.

58. The method of any one of the preceding claims, wherein the method further comprises repeating the anticancer therapy and repeating the administration of the suppressing agent.

59. The method of any one of the preceding claims, wherein the method further comprises determining if an adverse event occurs.

60. The method of any one of the preceding claims, wherein the subject is treated with antibiotics prior to administration of the suppressing agent.

61. The method of any one of the preceding claims, wherein multiple doses of the suppressing agent are administered.

62. The method of any one of the preceding claims, wherein the suppressing agent is administered after the completion of one round of the anticancer therapy.

63. The method of any one of the preceding claims, wherein the suppressing agent is administered at least one week, at least two weeks, at least three weeks or at least twelve weeks after the completion of one round of the anticancer therapy.

64. The method of any one of the preceding claims, wherein the suppressing agent is administered prior to the completion of one round of the anticancer therapy.

65. The method of any one of the preceding claims, wherein the suppressing agent is administered at least one week, at least two weeks, at least three weeks or at least twelve weeks after the initiation of the anticancer therapy.

66. The method of any one of claims 59-65, wherein determining if an adverse event occurs includes determining if IL-17 is increased in serum of the subject.

67. The method of any one of claims 59-66, wherein determining if an adverse event occurs includes testing for eosinophilia.

68. The method of any one of the preceding claims, wherein the subject is being treated for melanoma, non-small cell lung cancer (NSCLC), Hodgkin's lymphoma, head and neck cancer, renal cell cancer, bladder cancer, or Merkel cell carcinoma.

69. The method of any one of the preceding claims, further comprising administering a steroid as a second suppressing agent.

70. The method of claim 69, wherein the steroid is prednisone.

71. The method of any one of the preceding claims, wherein the suppressing agent comprises infliximab.

72. The method of any of the preceding claims, further comprising administering infliximab as a second suppressing agent.

73. A composition comprising the anticancer agent of any one of the preceding claims and the suppressing agent of any one of the preceding claims.

74. A kit comprising an agent for detecting a biomarker for an adverse event and the suppressing agent of any one of the preceding claims.

75. A kit comprising the anticancer agent of any one of the preceding claims, an agent for detecting a biomarker for an adverse event and the suppressing agent of any one of the preceding claims.
